SEQUENCE LISTING (31 sequences)

SEQ ID NO: 1 - protein, 416 aa, Arabidopsis thaliana
GSHMAEEKWVVMVTAQTPTNIAVIKYWGKRDEVRILPINDSISVTLDPDHLCTLTTVAVSPSFDRDRMWLNGKEISLSGSRYQNCLREIRSRADDVEDKEKGIKIAKKDWEKLHLHIASHNNFPTAAGLASSAAGFACLVFALAKLMNVNEDPSQLSAIARQGSGSACRSLFGGFVKWNMGNKEDGSDSVAVQLVDDKHWDDLVIIIAVVSSRQKETSSTSGMRESVETSLLLQHRAKEVVPVRILQMEEAIKNRDFTSFTKLTCSDSNQFHAVCMDTSPPIFYMNDTSHRIISLVEKWNRSAGTPEIAYTFDAGPDHAVMIARNRKVAVELLQGLLYCFPPKPDTDMKSYVLGDTSIVKEAGLEGELPQGIKDKIGSQDQKGEVSYFICSRPGRGPVVLQDQTQALLHPQTGLPK

SEQ ID NO: 2 - protein, 413 aa, Arabidopsis thaliana
MAEEKWVVMVTAQTPTNIAVIKYWGKRDEVRILPINDSISVTLDPDHLCTLTTVAVSPSFDRDRMWLNGKEISLSGSRYQNCLREIRSRADDVEDKEKGIKIAKKDWEKLHLHIASHNNFPTAAGLASSAAGFACLVFALAKLMNVNEDPSQLSAIARQGSGSACRSLFGGFVKWNMGNKEDGSDSVAVQLVDDKHWDDLVIIIAVVSSRQKETSSTSGMRESVETSLLLQHRAKEVVPVRILQMEEAIKNRDFTSFTKLTCSDSNQFHAVCMDTSPPIFYMNDTSHRIISLVEKWNRSAGTPEIAYTFDAGPDHAVMIARNRKVAVELLQGLLYCFPPKPDTDMKSYVLGDTSIVKEAGLEGELPQGIKDKIGSQDQKGEVSYFICSRPGRGPVVLQDQTQALLHPQTGLPK

SEQ ID NO: 3 - protein, 426 aa, synthetic construct
MGSSHHHHHHSSGMAEEKWVVMVTAQTPTNIAVIKYWGKRDEVRILPINDSISVTLDPDHLCTLTTVAVSPSFDRDRMWLNGKEISLSGSRYQNCLREIRSRADDVEDKEKGIKIAKKDWEKLHLHIASHNNFPTAAGLASSAAGFACLVFALAKLMNVNEDPSQLSAIARQGSGSACRSLFGGFVKWNMGNKEDGSDSVAVQLVDDKHWDDLVIIIAVVSSRQKETSSTSGMRESVETSLLLQHRAKEVVPVRILQMEEAIKNRDFTSFTKLTCSDSNQFHAVCMDTSPPIFYMNDTSHRIISLVEKWNRSAGTPEIAYTFDAGPDHAVMIARNRKVAVELLQGLLYCFPPKPDTDMKSYVLGDTSIVKEAGLEGELPQGIKDKIGSQDQKGEVSYFICSRPGRGPVVLQDQTQALLHPQTGLPK

SEQ ID NO: 4 - protein, 413 aa, Lepidium sativum
MAEEKWVLMVTAQTPTNIAVIKYWGKRDEVRILPINDSISVTLDPEHLCTLTTVAVSPSFDRDRMWLNGKEISLSGSRYQNCLREIRSRADDVEDKVKGIKIAKKYWEKLHLHIASHNNFPTAAGLASSAAGFACLVFALAKLMNVDEDASQLSAIARQGSGSACRSLFGGFVKWNMGNKEDGSDSIAVQLADDKHWDDLVIIIAVVSSRQKETSSTSGMRESVETSLLLKHRAKEIAPVRILQMEEAIKNRDFTSFTKLTCADSNQFHAVCMDTSPPIFYMNDTSHRIISLVEKWNRSAGTPEIAYTFDAGPDHAVLIARNRKVAVELLQGLLYCFPPQPDTDLKSYVLGDTSIVKEAGLEREIPQGIKDKIGSQDKKGEVSYFICSRPGKGPVVLQDQAQALLHPQTGLPK

SEQ ID NO: 5 - protein, 415 aa, Thlaspi arvense
MAEEKWVLMVTAQTPTNIAVIKYWGKRDEVRILPINDSISVTLDPDHLCTLTTVAVSPAFDRDRMWLNGKEISLSGSRYQNCLREIRSRGDDVEDEAKGIKIAKKDWEKLHLHIASYNNFPTAAGLASSAAGFACLVFALAKLMNVHEDPSQLSAIARQGSGSACRSLFGGFVKWNMGNKEDGSDSVAVQLADEKHWDDLVIIIAVVSSREKETSSTSGMRESVETSLLLQHRAKEVVPVRMLQMEEAIKNRDFTSFTKLTCSDSNQFHAVCMDTSPPIFYMNDTSHRIISLVEKWNRTVGTPEIAYTFDAGPDHAVLIARSRKVAVELLQGLLYCFPPKPDTDMKSYVLGDTSIVKEAGLERGGEAPQEIKDKIGSQDQKGEVSYFICSRPGKGPVVLQDQTQALLHPETGLPK

SEQ ID NO: 6 - protein, 420 aa, Brassica napus
MAAEKWVYMVTAQTPTNIAVIKYWGKRDEVRILPVNDSISVTLDPDHLCTVTTVAVSPSFDRDRMWLNGKEISLSGSRYQNCLREIRSRGGDVEDKEKGVKIEKKDWERLNLHIASHNNFPTAAGLASSAAGFACLVFSLAKLMNVEEDPSQLSAIARQGSGSACRSLFGGFVKWKMGSKDDGSDSVAVQLADEKHWDDLVIIIAVVSSRQKETSSTSGMRESVETSLLLQHRAKEVVPKRIVQMEEAIKNRDFASFTQLSCTDSNQFHAVCLDTSPPIFYMNDTSHRIISLVEKWNRSEGTPQVAYTFDAGPDHAVLIARNRKVAVQLLQGLLYYFPPKSDTDMKSYVVGDNSILKEAGLDGENSVENLQPPPEIRDNIGSQDQKGEVSYFICTRPGRGPVVLPDQTQALLDPETGLPK

SEQ ID NO: 7 - protein, 420 aa, Cakile maritima
MAAEKWVHMVTAQTPTNIAVIKYWGKRDEVRILPVNDSISVTLDPDHLCTVTTVAVSPAFDRDRMWLNGKEISLSGSRYQTCLREIRSRGGDVEDKEKGVKIEKKDWERLNLHIASHNNFPTAAGLASSAAGFACLVFSLAKLMNVDEDPSQLSAIARQGSGSACRSLFGGFVKWTMGSKEDGSDSVAVQLADEKHWDDLVIIIVVVSSRQKETSSTSGMRESVETSLLLQHRAKEIVPKRIVQMEEAIKTRDFASFTQLSCTDSNQFHAVCLDTSPPIFYMNDTSHRIISLVEKWNRSEGTPQVAYTFDAGPDHAVLIARNRKVAVQLLQGLLYYFPPKSDTDMKSYVVGDNSILKEAGLDGANSVENLQPPPEIKDNIGTQDQKGEVSYFICTRPGRGPVVLPDQTKALLDPETGLPK

SEQ ID NO: 8 - protein, 422 aa, Juglans regia
MVGGAEKWVMMVTAQTPTNIAVIKYWGKRDESLILPVNDSISVTLDPDHLCTTTTVAVSPSFDRDRMWLNGKEISLSGGRYQNCLREIRSRASDVEDEENGIKIPKKDWEKLHLHIASYNNFPTAAGLASSAAGFACLVFSLAKLMNVKEDQSKLSAIARQGSGSACRSLYGGFVKWIKGRDEDGRDSLAVQLADEKHWDDLLIIIAVVSSRQKETSSTTGMCETVETSLLLKHRAKEIVPKRILQMEEAIKNRDFTSFAQLACADSNQFHAVCLDTSPPIFYMNDTSHRIISIAEKWNRSEGTPQVAYTFDAGPDHAVLIARDRKAATLLLQRLLFYFPPSSATDLDSYVLGDKSILKDAGIEGIIDVEGLPPPPEIKDSIPTQKYKGDVSYFICTRPGRGPVLLSDKSQALLNPQDGLPK

SEQ ID NO: 9 - protein, 420 aa, Carica papaya
MADKWVLMVTAQTPTNIAVIKYWGKRDETLILPINDSISVTLDPDHLCTTTTVAVSPVFDQDRMWLNGKEISLSGGRYQNCLREIRSRAGDVEDKEKGIKIVKEDWKKLHVHIASYNNFPTAAGLASSAAGFACLVFALANLMNVKEDQSKLSAIARQGSGSACRSLFGGFVKWIMGQDENGSDSIAVQLADEKHWDDLVIIIAVVSSRQKETSSTSGMRESVETSLLLQHRAKEIVPKRIQEMEEAIKSCDFASFAQLTCADSNQFHAVCLDTCPPIFYMNNTSHRIISIVEKWNRSEGTPQVAYTFDAGPDHAVLIARDRNVATQLLQRLLFYFPPTSDTDLSSYVLGDKSILGEAGLEGIKDVEALQPPPETKDNSISAQKYKGDVSYFICTRPGRGPVLLSDSNHALLNPETGLPK

SEQ ID NO: 10 - protein, 425 aa, Cucurbita moschata
MAISRESEGGKWVLMTTAQTPTNIAVIKYWGKRDEALILPVNDSISVTLDPSHLCTITTVAVSPDFEKDRMWLNRKEISLSGARYQNCLREIRSRANDVEDKEKGIKIAKKDWEKLHVHIDSYNNFPTAAGLASSAAGFACLVFALANLMNVKEDHSQLSAIARQGSGSACRSLYGGFVKWSMGKEKDGSDSLAIQLADEKHWDDLVIIIAVVSSRQKETSSTSGMRETVETSLLLQHRAKEVVPKRVLAMEEAIQNRDFVSFAQLTCNDSNQFHAVCLDTSPPIFYMNDTSHRIISLVEKWNHSEGEPQVAYTFDAGPDHSVLIARNRKTAVSLLQRLLFQFPPNPETELNSYVLGDKTILQDAGINSVEDIESLPQPPEMSSSFQKYQGDVSYFICTRPGKGPVVLPESEALLDPKTGLPKNL

SEQ ID NO: 11 - protein, 421 aa, Coffea arabica
MAEKKSWVLMVTAQTPTNIAVIKYWGKRDESLILPINDSISVTLDPSHLCTTTTVAVSPGFTHDRMWLNGKEISLSGGRYQNCLREIRSRASDVEDEKKGIKIAKKDWENLHLHIASYNNFPTAAGLASSAAGFACLVYSLAKLMNVKEDNSQLSAIARQGSGSACRSLYGGFVKWIMGKDDNGSDSIAVQLVDEKHWDELVILIAVVSSRQKETSSTSGMRETVETSPLVLHRAKEVVPKRVVEMEEAIKSRNFPAFARLTCSDSNQFHAVCLDTSPPIFYMNDTSHRIISCVEKWNRSEGTPQVAYTFDAGPDHAVLIARNRKVAALLLQRLLFHFPPQSDADLNSYIVGDKSVLMDAGIQDLKDVEALPPPPEIKDNVPTQKFKGDVSYFICTRPGRGPVLLTEESQALLNPETGLPK

SEQ ID NO: 12 - protein, 433 aa, Capsicum chinense
MAEQSKKWVLMVTAQTPTNIAVIKYWGKRDENLILAINDSISVTLDPAHLCTTTTVAVSPDFDEDRMWLNGKEISLAGGRYQNCLREIRARASDVEDEKKGIKITKNDWQNLHVHVASYNNFPTAAGLASSAAGFACLVFSLAKLMNVQEDNGRLSAIARQGSGSACRSLFGGFVKWVMGKEEDGSDSIAVPLVDEKHWDELVIIIAVVSSRQKETSSTSGMRDTVETSSLIEHRAKEVVPKRIIKMEEAIQNRDFPTFAQLTCSDSNQFHSVCMDTTPPIFYMNDTSHRQKVLTGCQLFRIIGCVEKWNRSEGTPQVAYTFDAGPDHAVLIARNRKAATLMLQRLLFHFPPNSDTDLNSYVIGDKSILKDAGIQDIKDVDALPPPPEIKDKIPAQKYKGEISYFICTRPGRGPVLLTDESQALLNPETGLPK

SEQ ID NO: 13 - protein, 422 aa, Oryza sativa
MAAAEEGQWVLMATGRSPTNIAVIKYWGKRDEALILPVNDSISVTLDPDHLSATTTVAVSPSFPSDRMWLNGKEISLSGGRFQSCLREIRKRAQDVEDEKKGIRIKKEDWGKLHVHIASFNNFPTAAGLASSAAGLACFVFTLGKLMNVKEDHGELSSIARQGSGSACRSIYGGFVKWCMGKNNDGSDSIAVQLADEAHWNDLVIIIAVVSSKQKETSSTSGMRDSVETSPLLQYRAQTVVPERVLKMEEAIKSRNFESFARLTCADSNQFHAVCLDTSPPIFYMNDTSHRIISLVEKWNQSEGTPQVAYTFDAGPDHAVLIAPNRKNATILLQKLLYYFPPQDNDLSSYMVGDKSILSDAGLKSIEDVEALPAPAETKMPSQKFKGDVSYFICSRLGAGPKVVTDESLALIDSVTGLPKGV

SEQ ID NO: 14 - protein, 389 aa, Glycine max
MASESQNWVFMVTAQTPTNIAVIKYWGKRDETLILPVNDSISLTLDPSHLCTTTTVAVSSAFHQDRMWLNAKEISLSGGRFQSCLREIRARACDVEDENKGIKITKEDWAKLHLHIASYNNFPTAAGLASSAAGFACLAYALGKLMDVKEDESQLSAIARQGSGSACRSLFGGFVKWIMGKEDNGSDSLAVQLADEKHWDDLVIVIAVVSSRQKETSSTSGMCESVETSLLLQHRAKFHAVCLDTSPPIFYMNDTSHRIISIAEKWNRSEEAPQVAYTFDAGPDHAVLIARNRKAATSLIQRLLYYFPPNSDDLSSYIIGDKSIAKDAGINGIQDVEALPPPPEIKDNIPPQKYKGDVSYFICTRPGRGPVLLSDSSQALLNGETGLPK

SEQ ID NO: 15 - protein, 7 aa, synthetic construct
  VARIANT 1: note = V, I, L, or F
  VARIANT 2: note = I or L
XXKYWGK

SEQ ID NO: 16 - protein, 6 aa, synthetic construct
  VARIANT 1: note = V, I, or L
XKYWGK

SEQ ID NO: 17 - protein, 6 aa, synthetic construct
  VARIANT 1: note = V, I, L, or A
XKYWGK

SEQ ID NO: 18 - protein, 9 aa, synthetic construct
  VARIANT 2: note = T or K
  VARIANT 3: note = A or G
  VARIANT 4: note = A, T, or S
PXXXGLASS

SEQ ID NO: 19 - protein, 9 aa, synthetic construct
  VARIANT 2: note = T or K
  VARIANT 3: note = A or G
  VARIANT 4: note = A, T, or S
  VARIANT 6: note = Any amino acid
  VARIANT 7: note = A or S
PXXXGXXSS

SEQ ID NO: 20 - protein, 9 aa, synthetic construct
  VARIANT 2: note = Any amino acid
  VARIANT 3: note = A, S, or G
  VARIANT 4: note = A, T, or S
  VARIANT 6: note = Any amino acid
  VARIANT 7: note = A or S
PXXXGXXSS

SEQ ID NO: 21 - protein, 7 aa, synthetic construct
  VARIANT 1: note = Q, K, R, or E
  VARIANT 3: note = Any amino acid
  VARIANT 4: note = Any amino acid
QKXXSST

SEQ ID NO: 22 - protein, 7 aa, synthetic construct
  VARIANT 1: note = Q, K, R, or E
  VARIANT 3: note = Any amino acid
  VARIANT 4: note = Any amino acid
  VARIANT 5: note = Any amino acid
QKXXXST

SEQ ID NO: 23 - protein, 7 aa, synthetic construct
  VARIANT 1: note = Q, K, R, or E
  VARIANT 3: note = Any amino acid
  VARIANT 4: note = Any amino acid
  VARIANT 5: note = Any amino acid
  VARIANT 7: note = T, S, or R
QKXXXST

SEQ ID NO: 24 - protein, 16 aa, synthetic construct
  VARIANT 4: note = Any amino acid
IARXGSGSACRSLFGG

SEQ ID NO: 25 - protein, 16 aa, synthetic construct
  VARIANT 11: note = Any amino acid
PPIFYMNDTSXRIISL

SEQ ID NO: 26 - protein, 10 aa, synthetic construct
PPIFYMNDTS

SEQ ID NO: 27 - protein, 9 aa, synthetic construct
VAYTFDAGP

SEQ ID NO: 28 - protein, 10 aa, synthetic construct
  VARIANT 2: note = Any amino acid
  VARIANT 3: note = Any amino acid
  VARIANT 4: note = Any amino acid
  VARIANT 5: note = Any amino acid
LXXXXTGLPK

SEQ ID NO: 29 - protein, 5 aa, synthetic construct
TGLPK

SEQ ID NO: 30 - protein, 396 aa, Saccharomyces cerevisiae
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 31 - protein, 408 aa, synthetic construct
MGSSHHHHHHSSGTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

(19) United States
(12) Patent Application Publication
(10) Pub. No.: US 2026/0240197 A1
(43) Pub. Date: Aug. 20, 2026

(54) Naturally occurring decarboxylase proteins with superior gelation properties for preparing foods and cosmetics

(71) Applicant: Shiru, Inc., Alameda, CA (US)

(72) Inventors: Christopher Farwell, RIchmond, CA (US); Eyal Akiva, Tel Aviv (IL); Ryan Leverenz, Kensington, CA (US); Janelle Myers, Berkeley, CA (US); Akemi Kunibe, San Francisco, CA (US); Prasanna Vasudevan, New Orleans, LA (US); Jason Voogt, Lafayette, CA (US); Jasmine Hume, Orinda, CA (US)

(73) Assignee: Shiru Inc., Alameda, CA (US)

(21) Appl. No.: 19/218,606

(22) PCT Filed: Sep. 29, 2023
(86) PCT No.: PCT/US2023/075601

§ 371 Date: May 26, 2025

Related U.S. Application Data
(60) Provisional application No. 63/411,112, filed on Sep. 29, 2022.

(51) Int. Cl. A23J 1/00; A23J 3/22; C12N 9/88

(52) U.S. Cl. CPC A23J 1/006; A23J 3/22; C12N 9/88; C12Y 401/01033

(57) ABSTRACT

This disclosure provides a structurally related family of gelation promoting decarboxylase homologs for use in commercial food products and cosmetics. GPDH proteins are expressed intracellularly in trace amounts in plants, animals, and eukaryotic microbes, where they play a catalytic role in the mevalonate pathway. Members of the GPDH family are structurally related by a series of closely conserved amino acid sequence motifs. The ability of GPDHs to promote gelation when used as an ingredient in commercial products was previously unknown. In contrast to most commonly used plant proteins and plant protein isolates, the combination of low gelation onset temperature (~50° C.) and a remarkably low critical gelation concentration (4%) makes the GPDH proteins of this disclosure particularly well suited as a functional protein replacement in foods and personal care products.

### PREVIOUS APPLICATION

This application claims the priority benefit of U.S. Provisional Application 63/411,112, filed Sep. 29, 2023. The priority application is hereby incorporated herein by reference in its entirety for all purposes.

### FIELD OF THE INVENTION

This patent disclosure relates generally to the identification of natural sources of new product ingredients. It provides a family of plant and microbe derived proteins with improved gelation properties suitable for use in commercial foods, cosmetics, and other manufactured products.

### BACKGROUND

There is a rapidly growing market for foods and cosmetics that are more ethically produced, sustainable and nutritious to improve the health of consumers and the planet.

The food industry is currently undergoing a revolution, as companies pivot toward the creation of a new generation of plant-based products to meet this consumer demand. There is an emphasis on developing plant-based foods that mimic traditional foods such as meat, fish, eggs, dairy foods, and products derived therefrom. A key challenge is to find ingredients from botanical or microbial sources that simulate the desirable appearance, texture, flavor, mouthfeel, and functionality of traditional foods. Texture and mouthfeel of meat and dairy substitutes are partly a function of gelation.

The cosmetic and beautification industries are undergoing their own revolution towards more sustainable ingredient sourcing and manufacturing. Objectives include reducing the content of synthetic ingredients, relying on naturally occurring ingredients rather than synthetics, and reformulating products to use less water and energy in their manufacture. The challenge in this context is to find naturally occurring ingredients for cosmetics that achieve target properties that are currently imparted by synthetics.

### SUMMARY

This disclosure provides a structurally related family of gelation promoting decarboxylase homologs (GPDHs) for use in commercial food products and cosmetics. GPDH proteins are expressed intracellularly in trace amounts in plants, animals, and eukaryotic microbes, where they play a catalytic role in the mevalonate pathway.

Members of the GPDH family are structurally related by a series of amino acid sequence motifs that are closely conserved across GPDH proteins from a wide spectrum of biological sources. A prototype GPDH is Shiru Gelation Protein SGP2A, which is diphosphomevalonate decarboxylase MVD1 from Arabidopsis thaliana. Another prototype GPDH is Shiru Gelation Protein SGP2B, which is diphosphomevalonate decarboxylase MVD1 from Saccharomyces cerevisiae S288C.

The ability of GPDHs to promote gelation as a food or cosmetic ingredient was previously unknown. The data provided in this disclosure establish that recombinantly produced and purified GPDHs have superior properties compared with commonly used plant proteins and isolates: specifically, reliable purity, low gelation onset temperature (~50° C.), low critical gelation concentration (4%), and virtually no melt-back. Naturally occurring GPDHs that are already in the food chain as part of an ingredient should face fewer regulatory hurdles as food additives. For all these reasons, GPDHs are well suited to replace gelling and texturizing components in meat, dairy, and egg alternative products, and in cosmetics.

Food Products and their Preparation

Disclosed in more detail below are food products, ingredients, and additives that contains a gelation promoting decarboxylase homolog (GPDH) at a concentration of 0.2% to 10% or 1 to 20% by weight of dry ingredients in the food product or ingredient. This does not include intact organisms in which the GPDH naturally occurs, such as yeast that may be added to the food for other reasons. The distinction is made by specifying that the GPDH is recombinantly produced, the GPDH has a non-naturally occurring sequence, or the product or additive has less than 1%, 2%, or 5% of other proteins from the organism in which a GPDH having substantially the same sequence is naturally expressed.

Also disclosed is a method of causing gelation or thickening of a food product during manufacture or upon heating, but including in the food product a purified or recombinant gelation promoting decarboxylase homolog (GPDH) at a concentration of 1 to 20% by weight of dry ingredients in the food product.

Also disclosed is a method of manufacturing a food product that is devoid of animal products, by recombinantly expressing a GPDH, optionally purifying the recombinant GPDH from other proteins produced by the host cell; and then preparing a food product containing 1 to 20% by weight of said recombinantly expressed GPDH.

Also disclosed is a method of preparing or improving a food product (for example, to increase nutritional value and/or reduce the environmental footprint) in a standard recipe by using a GPDH to replace one or more previously used gelation or thickening agents: for example, methylcellulose, carboxymethylcellulose (CMC), pectin, gums such as agar, xanthan gum, guar gum, locust bean gum, carrageenan), starches, tapioca, and proteins obtained from chickpea, pea, fava bean, egg, milk, wheat, and gelatin.

Included in any of these products and methods are the manufacture, distribution, and use of food products such as patties, meatballs, sausage, and chicken-like nuggets, plant-based ice cream, a flan or custard, a cake, a soup, and frozen or refrigerated dough.

A meat substitute, replacement, or replica is a food in which one or more animal meats is replaced with a plant or tissue-based component chosen to have similar texture and/or flavor. A meat substitute can contain, for example, a protein content of at least 10% by weight, wherein at least 75% of the protein content is a mixture of plant proteins and/or one or more products of tissue culture; and optionally a content of at least 5% by weight, wherein at least 75% of the fat content is one or more oils isolated from agricultural crops or cultures. Optionally, the protein content and the fat content form a muscle replica and a fat tissue replica that are assembled in the product in a manner that approximates the physical organization of meat.

After cooking, the meat substitute or a product made from the flavor additive preferably has a meat-associated aroma and/or taste. The meat-associated flavor may be imparted, for example, by including 0.2% to 5% by weight of a heme-containing protein or a porphyrin binding protein. The meat substitute often contains a sugar such as glucose, ribose, fructose, lactose, xylose, arabinose, glucose-6-phosphate, maltose, and galactose, and mixtures of two or more thereof. To further enhance flavor, the product may also include a protein-free sulfur-containing compound, such as cysteine, cystine, selenocysteine, thiamine, methionine, and mixtures of two or more thereof.

A plant-based GPDH containing ice cream typically has a protein content of at least 5% by weight, wherein at least 75% of the protein content is a mixture of plant proteins and/or one or more products of tissue culture; a fat content of at least 5% by weight, wherein at least 75% of the fat content is one or more plant derived oils; and a naturally occurring sweetener of at least 5% by weight (and/or an artificial sweetener, to give a desired degree of sweetness). When frozen at −5 to −25° C., the combination stays mixed and has the mouthfeel of an ice cream.

### Personal Care Products, Pharmaceuticals, and Other Uses

Disclosed in more detail below are cosmetics and ingredients of personal care product that contains a gelation promoting decarboxylase homolog (GPDH) at a concentration of 1 to 20%, 1 to 5%, or at least 5% by weight of the product or ingredient but less than 1%, 2%, or 5% of other proteins from an organism in which the GPDH is expressed naturally. Depending on context, the GPDH may texturize or thicken the product or ingredient, or promote or stabilize emulsification of the components thereof.

Also disclosed is a method of texturizing, thickening, or emulsifying a cosmetic product or personal care ingredient during manufacture, the method comprising including in the product or ingredient a purified or recombinant GPDH at a concentration of 1 to 20% by weight of the product or ingredient. Also disclosed is a method of improving a cosmetic product or personal care ingredient, comprising preparing the product using a recipe in which one or more previously used components thereof is replaced with a GPDH at a concentration of 1 to 20% by weight of the product or ingredient. For example, the previously used component may be hyaluronic acid (HA), methyl- or ethyl-cellulose, hydroxypropyl methylcellulose, a gum, a wax, or other currently used component listed below.

The cosmetic or personal care product may be a moisturizer, eye or skin makeup preparation, lipstick, lip balm, lotion, facial cleanser, pomade, shaving cream, oral hygiene product, facial treatment, skin care preparation, or a suntan or sunblock preparation. Besides or instead of texturizing, thickening, and emulsifying, the GPDH may have the effect of increasing or improving viscosity, color or color fixing, antibiotic activity, sun protection factor (SPF), water resistance, glossiness, stabilizing activity, moisturizing activity, film-forming, smoothness, lubricity, pearlescence, and physical structuring.

Also disclosed below are pharmaceutical and nutraceutical products that contains a pharmaceutically active agent or nutritious ingredient combined with a gelation promoting decarboxylase homolog (GPDH) as a pharmaceutically compatible excipient thereof, wherein the GPDH is present in the composition at a concentration of 1 to 20% by weight of dry ingredients. Also included are pharmaceutical and nutraceutical products that contain a pharmaceutically active agent or nutritional ingredient encapsulated in a capsule or particle, wherein the capsule or particle comprises a gelation promoting decarboxylase homolog (GPDH) at a concentration of 5 to 75% by weight.

More generally, the disclosure provides an industrial product for commercial sale or public use that contains a gelation promoting decarboxylase homolog (GPDH) at a concentration of 0.2 to 10%, 1 to 20%, 1 to 5% or at least 5% by weight of dry ingredients in the product but less than 1%, 2%, or 5% of other proteins from an organism in which the GPDH is expressed naturally. In principle, any industrial product that contains or would benefit from the presence of a gelation, texturizing, thickening, or emulsifying component could benefit by selection and optimization of a GPDH put forth in this disclosure.

### Characteristics of GPDHs

In any of the products and methods referred to above, each GPDH may have one, two, three, or more than three of the following structural and functional features in any combination: an amino acid sequence that contains any of the A and/or B and/or C and/or D and/or E and/or F and/or G motifs defined in FIG. 9B in any combination;
an amino acid sequence that is at least 60%, 70%, 80%, 90%, 95%, or 99% identical to the sequence of SGP2A (SEQ. ID NO: 1), SGP2B (SEQ. ID NO:30), or any other sequence of any one of a number of related proteins, such as SEQ. ID NOS:2 to 14, as determined by the BLAST algorithm (SF Altschul et al., 1990; J. Mol. Biol. 2015:403-410);
it has been produced by recombinant expression;
it contains the same sequence as a naturally occurring protein or a gelation promoting, thickening, or texturizing portion thereof, preferably expressed in non-vertebrates, non-animals, or by plants, fungi, or cyanobacteria;
it may or may not have diphosphomevalonate decarboxylase enzyme activity.

In any of these products and methods, the GPDH may have any of the desired gelation properties or functions put forward in this disclosure in any combination, including but not limited to: a critical gelation concentration of no more than 4%, 6%, or 8%;
a 12% (wt/wt) solution of the GPDH has a density of at least 1.1 g/cm³;
a gelation Tₒₙₛₑₜ of 55 to 75° C.; 40 to 70° C., or over 40, 50, or 60% a 12% (wt/wt) solution of the GPDH has an ultimate gel strength of at least 500 Pa or 2,000 Pa, or between 1,000 to 10,000 Pa;
a 12% (wt/wt) solution of the GPDH has an ultimate gel elasticity of 1 to 15% or at least 4% critical strain.

In any of these products and methods, the GPDH may be an Arabidopsis or yeast protein and/or have the folding or three dimensional structure of SGP2A or SPG2B, or other members of the diphosphomevalonate decarboxylase family. Typically, the GPDH is recombinantly produced and isolated before being added as an ingredient to the food product, cosmetic, or other product. The GPDH may have been fragmented, mutated, hydrolyzed, digested, denatured, crosslinked, conjugated to another substance, or otherwise industrially processed, either before or after inclusion in the substance or article of manufacture. Alternatively or in addition, the product has less than 0.2%, 1%, 2%, or 5% of other proteins from an organism in which the GPDH is expressed naturally.

Additional aspects, embodiments, features, and characteristics of the invention, its products, their manufacture, and their use are described in the sections that follow, the accompanying drawings, and the appended claims.

### BRIEF DESCRIPTION OF THE DRAWINGS

FIGS. 1A and 1B provide several depictions of the structure and physiological function of SGP2A, which is diphosphomevalonate decarboxylase MVD1 from Arabidopsis (Arabidopsis thaliana), EC 4.1.1.33. It is the prototype for the gelation promoting decarboxylase homologs (GPDHs) of this disclosure. FIG. 1C shows the known and predicted associations of MVD1 with other proteins. FIG. 1D lists homologs and orthologs of SGP2A in other plants.

FIG. 2A shows the appearance of one preparation lot of gelled SGP2A and the corresponding gelation heat maps. FIG. 2A also shows the gelation propensities of 12% w/w solutions of powder lot PL51B in food-relevant conditions, varying protein concentration, salt concentration, and pH.

FIG. 2B shows purity of recombinantly produced SGP2A in crude lysate or purified protein samples, as determined by SDS gel electrophoresis. FIG. 2C shows the appearance of a preparation lot of gelled SGP2B.

FIG. 3 is a flowchart showing a scheme whereby other GPDHs can be prepared and tested for relevant properties as food ingredients.

FIGS. 4A and 4B are plots of storage modulus (G′) vs temperature for SGP2A and SGP2B respectively, in comparison with other compounds typically used in foods for gelation: specifically, methyl cellulose, Solanic 200 potato protein isolate, and ovalbumin.

FIG. 4C shows graphically the effects of pH and sodium chloride concentration on the solubility of SGP2B in 1% wt/vol protein dispersions. Because SGP2B is highly soluble at pH 6 to 8, independent of salt concentrations, it is well suited for use in food formulations.

FIG. 4D summarizes gelation propensities of a preparation of SGP2B. In contrast to most commonly used plant proteins and plant protein isolates, SGP2 has a combination of low gelation onset temperature (~50° C.) and low critical gelation concentration (4%).

FIGS. 4E and 4F are images of meatball prototypes prepared using 2% (wt/wt) recombinant SGP2B, either before or after cooking.

FIG. 4G compares the hardness of meatballs containing recombinant SGP2A or SGP2B with meatballs containing methylcellulose. The recombinant proteins caused gelation of the ingredients during cooking, resulting in a well-textured cooked product.

FIG. 5 is a chart showing plant and animal proteins currently used in food industry and some of their food applications (adapted from JT Martins et al., Front. Sus. Food Sys. 2018, 2:77).

FIGS. 6A to 6C provide amino acid sequences of a prototype GPDH protein designated SGP2A (SEQ. ID NOS:1 to 3). FIGS. 6D and 6E provide amino acid sequences of a second GPDH sequence designated SGP2B (SEQ. ID NOS:30 and 31)

FIG. 7 is a comparison of the amino acid sequences of SGP2A (SEQ. ID NO: 1) and SGP2B (SEQ. ID NO:30). The highlighted regions are motifs that are closely shared throughout members of the GPDH family, even though the amino acid sequences of SGP2A and SBP2B are only about 40% identical to each other.

FIGS. 8A to 8K provides the amino acid sequence of several species and strain homologs of SGP2A (SEQ ID NOS:4 to 14).

FIG. 9A shows the domain configuration of certain diphosphomevalonate decarboxylases in the GPDH class. FIG. 9B shows amino acid motifs (SEQ ID NOS: 15 to 29) that were identified during the course of this project as shared features of GDPH proteins

FIG. 10 is a sequence similarity network of selected GPDHs, representing the degree of sequence identity between different diphosphomevalonate decarboxylases.

### DETAILED DESCRIPTION

This disclosure provides for the first time a family of gelation promoting decarboxylase homologs (GPDH) that can be used as gelation agents in food products. Some of the proteins in this category are enzymes: specifically, diphosphomevalonate decarboxylase, EC 4.1.1.33), Gelation properties of the GPDH family were previously unknown. GPDHs can be used in place of currently used gelation ingredients, having superior gelation performance and other beneficial properties.

1. Superior Properties of GPDHs of this Disclosure, Compared with Currently Used Relation Agents

The food development and cosmetic industries are seeking clean-label gelling and texturizing agents from non-animal sources that have better properties than agents currently used. Methylcellulose, the most frequently used gelation agent in plant-based meats, and other alternatives (such as polysaccharide gums) have negative consumer attitudes and questionable nutritional value. Gelling agents like methylcellulose can exhibit undesirable properties like “melt-back”, which is the pronounced loss of gel strength (or total loss of gel structure) that occurs upon cooling for a thermoreversible gel.

By contrast, the GPDHs of this disclosure are naturally occurring proteins that constitute a clean-label digestible protein source. Some GPDHs are natural plant products, which means that they are already a minor part of the existing food system. As a gelation-inducing food component, GPDHs are a nutritional upgrade over gums and cellulosic gelling ingredients. GPDHs have a low critical gelation concentration (<6%), low onset gelation temperature (Tₒₛₑₜ<70° C.), and absence of melt-back. GPDHs provide binding, viscosifying, texturizing, and moisture retaining functionalities in food products. GPDHs have potential in multiple food category applications, such as plant-based & cellular grown meats, bakery products, dairy products and their derivatives, beverages, soups, and sauces.

Previously known vegan alternatives to methylcellulose are citrus fiber, potato protein isolate, and canola protein isolate. Production of these alternatives may be adversely affected by monoculture or disease related supply fluctuations, or have inconsistent organoleptic properties (bitter or vegetal flavors). GPDHs can be manufactured by a closely controlled process, incorporating optimal energy usage, waste control and management, which provides a more reliable consistency of product. Recombinantly produced GPDHs have exceptionally high standards of homogeneity, inter-batch consistency, food safety, hygiene, and final ingredient quality.

2. Discovery of GPDHs and their Beneficial Properties as Food Ingredients

Gelation properties of the GPDH family were discovered by the characterization of prototype SGP2A. The prototype was identified as part of a project to identify gelation proteins that have desirable heat-onset hydrogelation characteristics.

As a first step in discovering new gelation-inducing agents, a selective starting database of protein sequences was created. Protein sequences were obtained from public databases, and screened by searching for a series of desirable characteristics. Then filters were applied to exclude proteins with undesirable characteristics. Three-dimensional structures were obtained from the protein data bank (PDB), compiled and made available from the Worldwide Protein Data Bank Foundation, Piscataway, NJ. To reduce the number of structural alignments to close variants, protein sequences that were redundant or fragments of other sequences in the database were removed, along with homologs with greater than 90% amino acid sequence identity. Proteins were also removed if they were known to interact with other proteins in a way that might complicate their expression, testing, or use as food ingredients. Otherwise, no restriction was made as to the natural biological function of each protein in the database. The curated dataset comprised a total of about 45,000 protein sequences.

To search the database for promising gelation candidates, 29 reference proteins were used that are known or that the inventors suspected of having beneficial gelation properties. Alignment of proteins in the database with each of the 29 reference proteins was done algorithmically in a pairwise fashion. Each reference protein produced a list of proteins with structural similarities with a p-value having a threshold for selection of <0.05. SGP2A was identified as having a degree of structural similarity to one of the 29 reference proteins. SGP2B was identified by cluster analysis of having superior gelation forming properties and being structurally related to SGP2A.

After identification, SGP2A and SGP2B were further evaluated to assess whether it could be empirically tested in the lab, and to confirm that it has no known toxic or allergenic properties. SGP2A was determined not to require any chaperones or post-translational modifications to fold properly. On this basis, it was selected as a candidate for recombinant expression and testing.

### 3. Role of SGP2A and SGP2B in Cell Biology

SGP2A and SGP2B are annotated in the UniProt and GenBank databases as isoforms of the enzyme diphosphomevalonate decarboxylase MVD1 from Arabidopsis thaliana (Mouse-ear cress) and Saccharomyces cerevisiae S288C (brewer's yeast), respectively. Diphosphomevalonate decarboxylase (EC 4.1.1.33), also referred to as mevalonate diphosphate decarboxylase, is an enzyme that catalyzes the chemical reaction via ATP dependent decarboxylation.

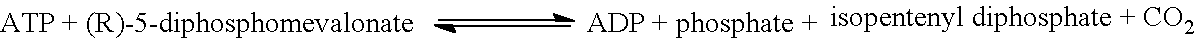

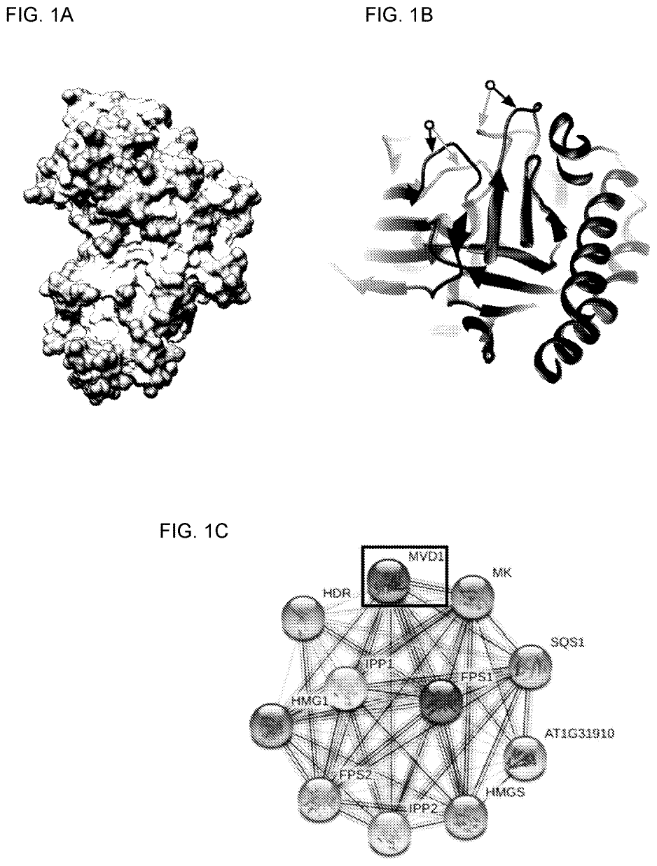

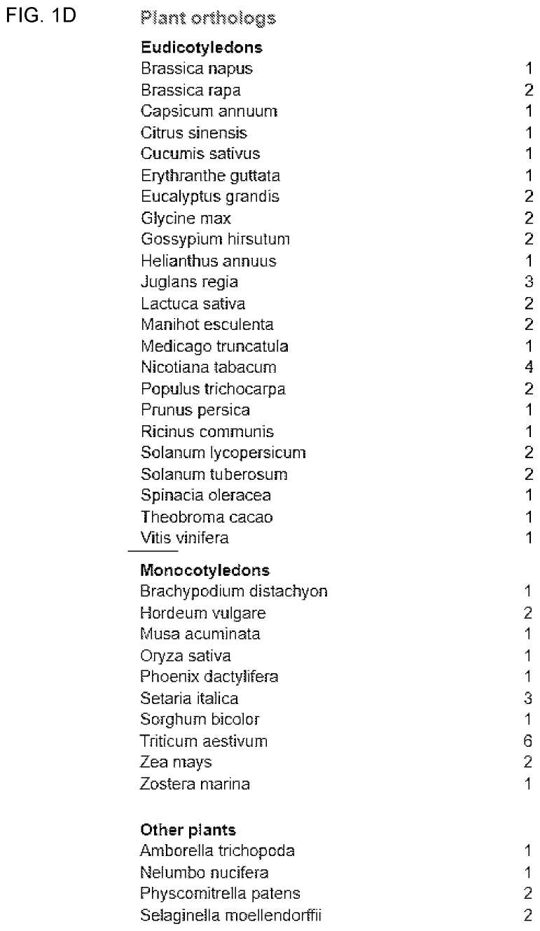

FIGS. 1A and 1B provide several depictions of the structure and physiological function of SGP2A, which is diphosphomevalonate decarboxylase MVD1 from Arabidopsis (Arabidopsis thaliana), EC 4.1.1.33. It is the prototype for the gelation promoting decarboxylase homologs (GPDHs) of this disclosure. FIG. 1C shows the known and predicted associations of MVD1 with other proteins. Data obtained from the STRING database, an ELIXIR Core Data Resource: R. Drysdale et al., F1000Research 2018, 7(ELIXIR):1711. FIG. 1D lists homologs and orthologs of SGP2A in other plants.

Mevalonate diphosphate decarboxylase catalyzes the final step in the mevalonate pathway. The mevalonate pathway is responsible for the biosynthesis of isoprenoids from acetate. The pathway plays a key role in multiple cellular processes by synthesizing sterol isoprenoids, such as cholesterol, and non-sterol isoprenoids, such as dolichol, heme A, tRNA isopentenyltransferase, and ubiquinone. The enzyme belongs to the family of lyases, specifically the carboxylyases, which cleave carbon-carbon bonds.

Many different organisms utilize the mevalonate pathway and mevalonate diphosphate decarboxylase, but for different purposes. ML Barta et al., Biochemistry. 51 (28): 5611-5621. In gram positive bacteria, isopentenyl diphosphate, the end product of mevalonate diphosphate decarboxylase, is an essential intermediate in peptidoglycan and polyisoprenoid biosynthesis.

The mevalonate pathway is also used in higher order eukaryotes and plants. Mevalonate diphosphate decarboxylase is mainly present in the liver of mammals where the majority of mevalonate is converted to cholesterol. Some of the cholesterol is converted to steroid hormones, bile acids, and vitamin D. Mevalonate is also converted into reaction intermediates, such as dolichols, ubiquinones, tRNA isopentenyltransferase and franesylated and geranylgeranylated proteins. DD Hinson et al., J. Lipid Res. 38 (11): 2216-23.

The ability of GPDHs to promote gelation when incorporated into a food, cosmetic, or other product as described here could not have been predicted from its previously known enzymatic and physiological roles.

4. The Science of Forming Gels from Food Ingredients and Assessing Gel Characteristics

The formation of food gels is a transformation of sol to the state of gel during which the viscoelasticity changes abruptly with simultaneous development of solid characteristics. There is an interchange of continuous and discontinuous phases during gel formation. TABLES 1A and 1B show experimental methods for determining gel characteristics (adapted from S. Banerjee et al., Crit. Rev. Food Sci. Nutr. 2012; 52:334-346).

TABLE 1A
Methods for the rheological measurement of gel characteristics

| Measurement | Instrument | Measurement parameters | Applications |
|---|---|---|---|
| Compression | Texture | Modulus of elasticity, Poisson's | Surimi gel |
| | measuring | ratio | |
| | system | | |
| Stress | Texture | Residual stress, relaxation time | Gellan gels |
| relaxation | measuring | | |
| | system | | |
| Creep | Controlled stress | Shear modulus, creep | Soy and gelatin gels |
| | rheometer | compliance | |
| Oscillation | Controlled stress | Storage modulus (G′), loss | Viscoelastic |
| | rheometer | modulus (G), phase angle, | characterization of |
| | | complex modulus, and viscosity | rice, soy gels, mixed |
| | | | gels |
| Puncture | Texture | Puncture characteristics | Characterization of |
| force | measuring | | rice gel |
| | system | | |
| Compression | Texture | Peak force, firmness, | Measurement of gel |
| | measuring | compression energy | quality and gel |
| | system | | strength |
| Texture | Texture | Parameters of texture and profile | Food gels |
| profile | measuring | analysis like hardness, | |
| analysis | system | brittleness, adhesiveness, | |
| (TPA) | | springiness, cohesiveness | |

These tests do not depend on the geometry of the sample and the instrument used. The rheological properties determined at large deformations include fracture, failure, and rupture characteristics like stress/strain, and are usually determined by uniaxial compression and tension. Rheological or textural characteristics of starch gels have been investigated mostly on model systems in order to understand basic factors and mechanism involved in the gelling and characterize the gel properties.

Rheological properties depend on the presence of molecular network. Measurement can be made which shows the relationship between stress (force per unit area) and strain (deformation due to applied force) for a gel under compression. The Young's or elastic modulus is the ratio of stress to strain of a material when tested within the linear limit of elasticity. The maximum stress that the gel can sustain is rupture strength (RS). Bulk modulus (K) can be obtained when the force is applied from all the directions (isotropically) and the change in volume per original volume is obtained.

TABLE 1B
Other methods for the measurement of gel characteristics

| Instrument used | Measurement parameters | Applications |
|---|---|---|
| Differential scanning calorimeter | Heat flow | Gellan and polyvinyl alcohol |
| (DSC) | | blend film |
| X-ray diffraction and SAXS | Particle size analysis | Nano delivery system in |
| | | food |
| Colorimeter | Color measurement | Gellan edible films |
| Light microscopy (LM) | Area of the granules | Tapioca starch gel |
| Scanning electron microscope | Structural arrangement of | The number, area and |
| (SEM) | components | location of particles in gel |
| Transmission electron | Structural distribution of | Characteristic studies of |
| microscopy (TEM) | constituents | mixed gel |
| Atomic force microscope | Structure of the molecules | Structural characteristics of |
| | | nanoparticles |
| NMR | Conformation changes on | Structural features of the |
| | gelation | constituents |
| FTIR | Molecular structure | Infrared spectra of the |
| | | components |
| FT-RAMAN; Near infrared | Molecular characterization | Functional characteristics of |
| resonance | | pectins |
| Vacuum oven | Moisture | Moisture estimation |
| Atomic absorption spectroscopy | Mineral content | Chitosan and whey protein |
| | | isolate based model system |
| Kjeldhal apparatus | Protein | Whey protein-cassava |
| | | starch gel |
| DNS method | Carbohydrate | Sugar content of the gel |

Different kinds of gel are formed depending on the molecular features and characteristics. For example, a hydrogel is a network of polymer chains that are water-insoluble, sometimes found as a colloidal gel in which water is the dispersion medium. An organogel is a non-crystalline, non-glassy, thermoreversible solid material composed of a liquid organic phase entrapped in a structuring network. A xerogel is a solid formed from a gel by drying with unhindered shrinkage, retaining high porosity and high surface area.

An aerogel is a colloidal gel in which gas is used as the dispersion medium. Some polysaccharides, often of food origin, show properties intermediate between polysaccharide solutions and true gels, and form weak gels. Under low deformation, weak gels behave as elastic gels. At sufficiently large deformation or at high shear rates, they fracture irreversibly, and flow. A fluid gel is formed when hot hydrocolloids dispersions are allowed to cool and set under quiescent condition. The dispersion separates into polymer-rich micro particles and polymer-poor regions, which forms the interstitial space between the particles.

### 5. General Methodology for Protein Expression and Purification

Typically, recombinant production of proteins such as GDPHs is done by genetic modification of a suitable expression host, genetically modified to integrate DNA or carry plasmids designed to express the protein of interest constitutively or via induction. Suitable organisms used for recombinant expression of candidate proteins are listed in TABLE 2. Host organism selection is done taking into consideration the ability for the host to express soluble protein in high quantities with the posttranslational modifications (such as addition of carbohydrates and/or interchain crosslinking) that may affect protein function.

TABLE 2
Recombinant expression systems for candidate proteins

| Organism | Strain |
|---|---|
| animal | Drosophila S2 |
| animal | SF9 |
| animal | SF21 |
| animal | CHO |
| yeast | Pichia pastoris (Komagataella phaffi) |
| yeast | Saccharomyces cerevisiae |
| filamentous fungi | Aspergilllus |
| filamentous fungi | Trichoderma reesi |
| filamentous fungi | Neurospora crassa |
| bacteria | E. coli |
| plant | Nicotiana benthamiana |
| plant | Solanum lycopersicum |
| algae | Chlamydomonas reinhardtii |
| cell free | plant extract |
| cell free | bacteria extract |
| cell free | yeast extract |

Eukaryotic expression systems have the advantage of performing post-translational processing of protein candidates in a manner akin to what may be used naturally or for industrial production, such as glycosylation and interchain crosslinking. Prokaryotic expression systems have the advantage of being easy to implement and obtain high yield. It is possible to use several systems during development: for example, expression in E. coli for performing screening assays; and expression in eukaryotes for later stage development and testing. Some expression systems such as yeast are suitable for use in both stages.

Common purification methods include centrifugation, filtration, affinity chromatography, ion exchange chromatography, size exclusion chromatography, hydrophobic interaction chromatography, affinity capture, isoelectric precipitation, liquid-liquid phase separation (LLPS), lyophilization, and dialysis. One of these methods may be used as a single step or combined with other methods as needed to achieve a desired level of purity. Once achieved, the protein is processed by standard methods into a final condition that is compatible with characterization methods. For example, some assay methods may require powdered protein, while other characterization methods may require proteins in aqueous solution. Protein Purification, 2nd Ed., P. Bonner, 2018; and High-Throughput Protein Production and Purification, R. Vincentelli ed., 2019.

To facilitate protein purification for the purpose of initial testing of GPDH candidates, recombinant protein can be expressed with an exclusive tag for affinity binding. The tag can be any feature added to the protein during expression that can be used as a handle for affinity purification using a conjugate binding partner. Examples include amino acid sequences added internally or to either end of the naturally occurring protein sequence, and carbohydrates. After the tagged protein is immobilized on an affinity surface, fermentation byproducts can be washed away. Depending on the tag used, the purified target protein can then be eluted from the resin using competitive binding or a condition change, such as pH.

### 6. Production and Purification of GPDHs

GPDHs can be prepared for initial testing using standard E. coli expression and purification methods. For example, overexpression of SGP2A was done using a pET28a (+) vector with expression induced using a lactose inducible promoter system. Plasmids were constructed to include a C-terminal 6X-HIS tag for purposes of detection as well as purification.

To obtain protein for physicochemical characterization, strains containing the SGP2A encoding sequence were grown at 30° C. at a scale of 2 mL to 10 L using 24-deep well plates or shake flasks, depending on volume. Standard Luria Broth (LB) media with IPTG induction or commercially available auto-induction media (such as MagicMedia™) causes overexpression of the SGP2A. Cells were harvested approximately 24 h after induction via centrifugation. Cells were then suspended 50 mM phosphate buffer, 500 mM sodium chloride, pH 7.5 for cell lysis. Cells were lysed using different methods depending on the scale using standard protocols for BugBuster®, sonication, or pressure homogenization. Lysate was then centrifuged to remove cellular debris.

Solubilized protein was collected and clarified using filtration with a 0.45 μM cutoff. Protein was purified using immobilized metal affinity chromatography and separated from contaminants by collecting fractionated eluate using an elution gradient 0 to 250 mM imidazole over 8 column volumes. Fractions containing SGP2A were identified by SDS-PAGE analysis, and pooled together. Protein volume was concentrated approximately 10 fold using tangential flow filtration cassettes with a molecular weight cutoff of 10 kDa. Salt was then removed from SGP2A by exchanging buffer to 18.2 megohm ionic strength water at a ratio of 1:40 for four successive exchanges using a method such as dialysis. The protein was dried by lyophilization for subsequent characterization and use.

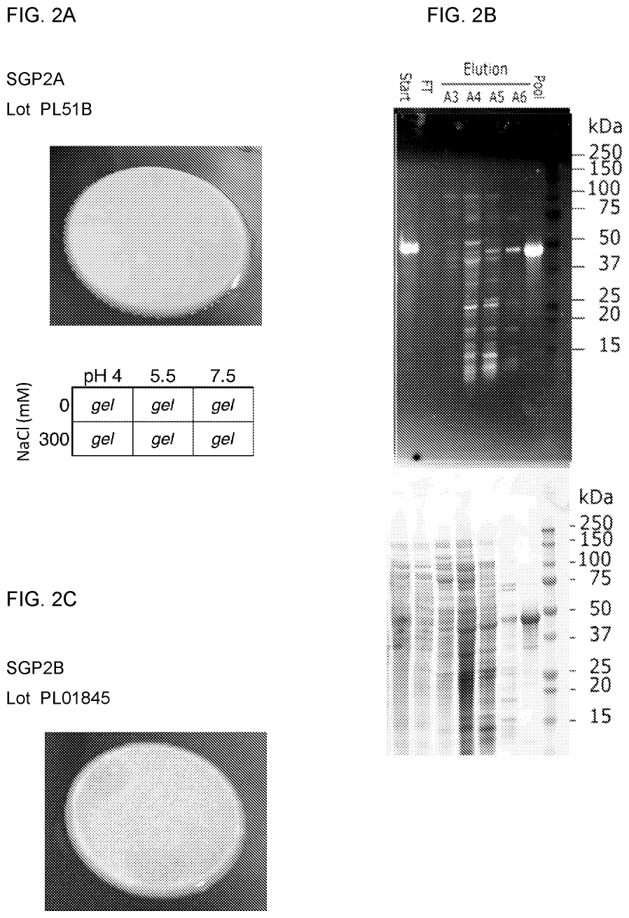

FIGS. 2A and 2C show the appearance of gelled preparations of purified SGP2A and SGP2B, respectively.

FIG. 2B shows purity of His-tag isolated protein SGP2A, as determined by SDS polyacrylamide gel electrophoresis. A primary single band was observed, corresponding to a mass at a mass that falls between 50 and 75 kDa.

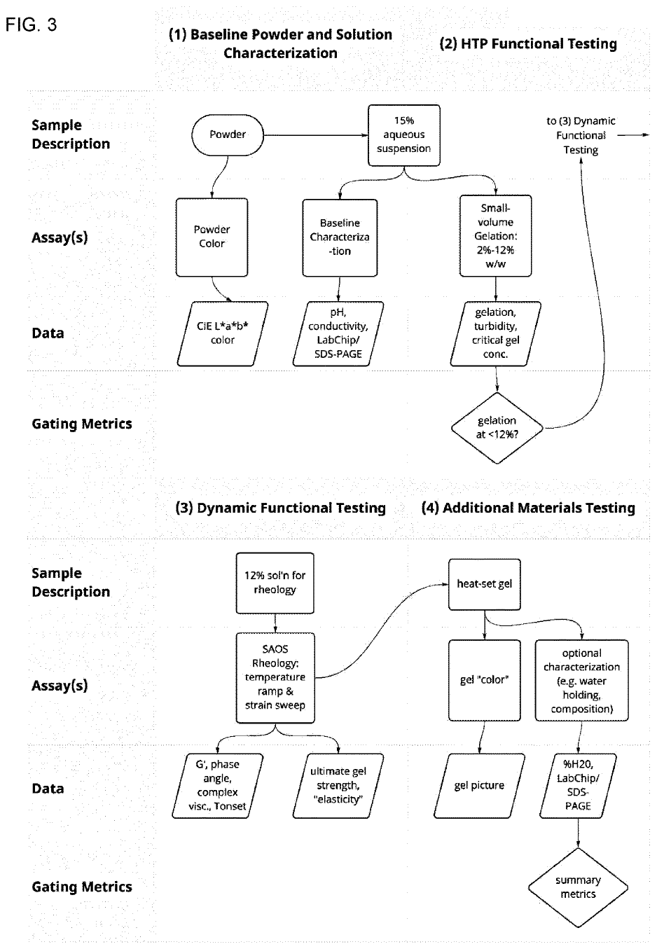

FIG. 3 is a flowchart showing a scheme whereby preparations of other GPDHs can be prepared and tested for relevant properties as food ingredients.

Production of protein SGP2A and other GPDHs can be scaled up for commercial manufacture for use as a food or cosmetic ingredient using other synthesis and purification protocols. Production methods may include changing the recombinant expression host to any one of the generally regarded as safe (GRAS) hosts, such as Pichia or Aspergillus. Protein can be produced via fermentation in a bioreactor, which can be sized for production of one to 10,000 liters. When used as a food ingredient, removal of artificial affinity tags is desirable, whereupon the protein is purified by other means. If protein is soluble and intracellular, cells must be lysed and cell debris must be removed, which can be done via centrifugation and filtration.

Protein can then be recovered via standard separation techniques such as fractionation, filtration, or a combination of separation techniques used in tandem. If protein is secreted from the host cell, then cell lysis may be omitted. Biochemical properties of the protein may be used to guide selection of such steps. For example, a molecular weight of 60 kDa can be used to select filtration methods with a molecular weight cutoff smaller than the protein, such that SGP2A remains in the retentate while filtration with a molecular weight cutoff greater than the protein such that the protein passes to the permeate. The isoelectric point of protein (calculated at 6 for protein SGP2A) can be used to guide pH at which the protein may become unstable and precipitate from solution. Such precipitation steps can be used to fractionate the GPDH from cellular debris and off-target proteins.

### 7. General Principles of Protein Characterization

Purified proteins that are GPDH candidates can be tested for various physicochemical properties, as listed in TABLE 3.

TABLE 3
Assessing biochemical properties

| Biochemical property | Assays |
|---|---|
| oligomerization state | size exclusion chromatography, native page |
| concentration | Bradford ™, Pierce 660 ™, absorbance |
| | spectroscopy |
| purity | amino acid analysis, proximate analysis, |
| | gel electrophoresis, capillary electrophoresis |
| buffering capacity | titration |
| pH | indicator strips, pH probe |
| enzyme activity | colorimetric assays, fluorometric assays, |
| | absorbance spectroscopy |
| molecular weight | gel electrophoresis, capillary electrophoresis |
| degradation | gel electrophoresis, amino acid analysis |
| conductivity | conductivity probe |
| % random coil | circular dichroism |
| % alpha helix | circular dichroism |
| % beta sheet | circular dichroism |
| zeta potential | phase analysis light scattering |
| solubility | fluorometric assays, colorimetric assays |
| aggregation | dynamic light scattering, centrifugation, |
| | size exclusion chromatography, |
| | fluorescence-based assays |
| particle size distribution | dynamic light scattering |
| melting temperature (tₘ) | differential scanning calorimetry, |
| | thermal shift assay |
| heat capacity | differential scanning calorimetry, |
| | thermal shift assay |
| surface hydrophobicity | fluorometric assay |
| % thiols | fluorometric assay |
| sulfur content | fluorometric assay |
| density biophysical | calculated |
| glycosylation content | mass-spectroscopy |

Purified proteins that are gelation candidates can be tested for various functional characteristics, as listed in TABLE 4.

TABLE 4
Assessing functional properties

| Functional property | Assays |
|---|---|
| gelation | rheology |
| aggregation | dynamic light scattering |
| texture | texture profile analysis |
| particle size | dynamic light scattering |
| viscosity | viscometry |
| sol gel transition temperature | rheology |
| denaturation temperature | differential scanning calorimetry |
| heat capacity | differential scanning calorimetry |
| chewiness | texture profile analysis |
| color | colorimeter |
| storage modulus | rheology |
| shear strength | rheology |
| density | densitometry |
| swell ratio wt/water | mass measurement |
| sedimentation layer thickness | emulsion stability analysis via multiple light scattering |
| sedimentation migration rate | emulsion stability analysis via multiple light scattering |
| emulsion stability index, | emulsion stability analysis via multiple light scattering |
| coalescing phase | |
| coalescence time | emulsion stability analysis via multiple light scattering |
| coalescing layer thickness | emulsion stability analysis via multiple light scattering |
| coalescence migration rate | emulsion stability analysis via multiple light scattering |
| emulsion stability index, | emulsion stability analysis via multiple light scattering |
| flocculating phase | |
| time to flocculation | emulsion stability analysis via multiple light scattering |
| flocculation layer thickness | emulsion stability analysis via multiple light scattering |
| flocculation migration rate | emulsion stability analysis via multiple light scattering |
| max foam volume | foam analysis via imaging |
| max liquid volume | foam analysis via imaging |
| gas volume foam | foam analysis via imaging |
| foam capacity | foam analysis via imaging |
| maximum foam density | foam analysis via imaging |
| foam expansion rate | foam analysis via imaging |
| foam half life time | foam analysis via imaging |
| drainage half life time | foam analysis via imaging |
| temperature at gelation point | rheology |
| yield stress | rheology |
| cohesiveness | texture profile analysis |
| adhesiveness | texture profile analysis |
| gumminess | texture profile analysis |
| melting point | differential scanning calorimetry |
| water binding capacity | moisture analysis |
| critical micelle concentration | dynamic light scattering |
| critical concentration for gelation | rheology |
| critical concentration for | moisture analysis |
| water binding | |
| antimicrobial action | microbial growth assays, fluorescent dye permeabilization, |
| | NMR spectroscopy |

### 8. Assessing Relation Characteristics of GPDHs

Particular assays were run on multiple preparations of SGP2A to test gelation characteristics by several criteria.

### Protein Thermal Shift Assay (TSA)

His-tag purified preparations of expressed SGP2A in solution were subjected to thermal shift analysis using differential scanning fluorimetry using the fluorophore SYPRO-Orange™ (ThermoFisher) and an Applied Biosystems 7500 RT-PCR thermal cycler with fluorescence reader, using excitation and emission wavelengths of 472 nm and 570 nm, respectively. Purified proteins that are gelation candidates can be tested for physicochemical properties. A protein sample was diluted to a concentration of 1 mg/mL in pure water and mixed with appropriate buffer and sodium chloride solution to obtain the pH and sodium chloride concentrations as shown in the table below, according to the BioRad protein thermal shift protocol. Buffer concentrations were 50 mM in all samples.

Analysis of protein melt curves was accomplished using in-house software and Tₘ was assigned based on the maximum of the derivative of the raw melt curve data. Results are shown in TABLE 5.

TABLE 5
Thermal shift properties of SGP2A

| NaCl (mM) | pH | Buffer | Tₘ (° C.) |
|---|---|---|---|
| 0 | 4.5 | citrate | denatured |
| 50 | 4.5 | citrate | denatured |
| 0 | 7.5 | phosphate | 50.8 |
| 50 | 7.5 | phosphate | 49.6 |

### Chemical and Physical Properties (Powder)

Following preparative-scale purification and lyophilization, the recovered SGP2A protein mass was ground to a powder with a mortar and pestle to generate a final protein/ingredient lot. Final lots were analyzed for color and composition, with results summarized in TABLE 6.

TABLE 6
Chemical and physical properties of SGP2A

| Property | Lot PL51B | Assay |
|---|---|---|
| measured color | (98.8, −0.10, 2.33) | reflectance |
| (L*, a*, b*) | | spectrophotometry |
| color description | bright white | qualitative |
| total protein (%) | 86.52% | PROT-HICOM |
| | | (Merieux NutriSciences) |
| total fat (%) | 0.12% | FAT-MOJOAH |
| | | (Merieux NutriSciences) |
| ash (%) | 1.55% | ASH |
| | | (Merieux NutriSciences) |
| moisture (%) | 5.80% | MOIST-VO |
| | | (Merieux NutriSciences) |

Powder color was measured by reflectance spectrophotometry (Konica Minolta CM-5) using ~0.2 g of powder loaded in a mini-petri dish for measurement. CIE L*a*b* color values are reported as the average of nine replicate measurements. Powder color may change with prolonged storage (for example, due to oxidation) or be correlated with certain compositional parameters (such as fat content) that can vary between lots.

### Solution Properties

Solubility of SGP2A and other GPDHs can be determined follows. Concentrated (15% wt/wt) SGP2A stock solutions are prepared by dissolving protein in ultrapure water. Solutions are gently homogenized by tube rotation and vortexing, followed by overnight storage. The pH and conductivity of the stock solution are measured in order to better quantify the extent of buffer salt removal following upstream diafiltration/dialysis and track lot-to-lot variation in this property.

To monitor lot-to-lot variations and provide quantitative data on solution turbidity, the stock solution is diluted (1:10) in water and the UV-visible spectrum (200-800 nm) collected using a NanoDrop™ spectrophotometer. Insoluble material is pelleted by benchtop centrifugation and the UV-visible spectrum (re)measured.

The primary structure, higher order structure, and structural homogeneity of SGP2A in the final powder lot may impact functional properties and performance. The homogeneity of denatured SGP2A is determined by measuring its apparent molecular weight on SDS-PAGE, or alternatively with a LabChip® protein express assay (SGP2A diluted 1:100 in 8 M urea).

### Gelation Properties

Thermal gelation of SGP2A protein solutions were initially tested using a qualitative gelation assay. Small volumes of protein solution are slowly heated to 92° C. in a temperature controlled water bath, and subsequently cooled to room temperature. Thermal gelation of protein solutions can also be assessed via inversion, resistance to puncture, and the ability to support a spherical mass. A solvent-dependent gelation heatmap was determined by assessing the gel-forming propensity of small volumes of 12% wt/wt protein solutions, buffered to pH 4 to 7.5 with 20 mM citrate/phosphate plus NaCl at concentrations of 0 to 300 mM. The critical gelation concentration was determined by measuring the gelation propensity of solutions of 0% to 12% SGP2A (wt/wt), dissolved in water or 20 mM phosphate pH 7.5, 300 mM NaCl). Results are shown in TABLE 7A. Similarly determined results for SGP2B are shown in TABLE 7B.

TABLE 7A
Gelation properties of SGP2A measured by rheological testing

| Property | Lot PL51B | Assay |
|---|---|---|
| critical gelation concentration | 4% in water | qualitative gelation |
| (wt/wt %) | | |
| gelation onset temp (Tₒₙₛₑₜ) | 50° C. | SAOS rheology |
| (approximated using dG′) | | |
| maximum gel strength, | 600.9 | SAOS rheology |
| temperature ramp (Pa) | | |
| ultimate gel strength (Pa) | 2,127.43 | SAOS rheology |
| ultimate gel elasticity | 2.46 | SAOS rheology |
| (critical strain, %) | | |

TABLE 7B
Gelation properties of SGP2B measured by rheological testing

| Property | Lot PL51B | Assay |
|---|---|---|
| critical gelation concentration | 4% in water | qualitative gelation |
| (wt/wt %) | | |
| gelation onset temp (Tₒₙₛₑₜ) | 55° C. | SAOS rheology |
| (approximated using dG′) | | |
| maximum gel strength, | 2241.7 | SAOS rheology |
| temperature ramp (Pa) | | |
| ultimate gel strength (Pa) | 8800 | SAOS rheology |

FIG. 2A shows the appearance of lot PL51B 1 of SGP2A in gel form, and the gelation heatmap at 12% (wt/wt) in solution. In all conditions tested, the qualitative appearance of the SGP2A gel was similar to the appearance of egg white. While no significant pH dependent effects on solubility or gelation were observed, the isoelectric point (6.33) of SGP2A lies within a pH range (pH 3-8) relevant to most food systems. The SGP2A protein may become less table near its isoelectric point. Thus, some variability in SGP2A solubility and/or the SGP2A heat-set gel structure may occur within this pH range. For a discussion of particulate versus strand-type gels, the reader is referred to Y. Cao and R. Mezzenga, Nature Food 2020, 106:118.

Small-amplitude oscillatory shear (SAOS) rheology was used to determine the dynamic rheological properties of the SGP2A solution/gel as a function of temperature. M. H. Tunick, J. Agric. Food Chem. 2011, 59:1481-1486, doi.org/10.1021. Data was collected using an Anton Paar MC302 model strain-controlled rheometer with PP25 (25 mm) parallel plate measuring system operating under small deformation conditions (0.1% strain at a constant angular frequency of 10 rad/sec). Temperature-dependent rheological properties were measured in 3 phases: (1) heating from 30° C. to 95° C. at a 5° C./min rate, (2) holding at 95° C. for 5 min, (3) cooling from 95° C. to 50° C. at 5° C./min rate. Following Phase 3 (cooling), a strain-amplitude sweep (0.01-100% strain) was carried out at a constant frequency (10 rad/sec) in order to probe the gel's linear viscoelastic region and shear-induced breakdown of the gel's superstructure.

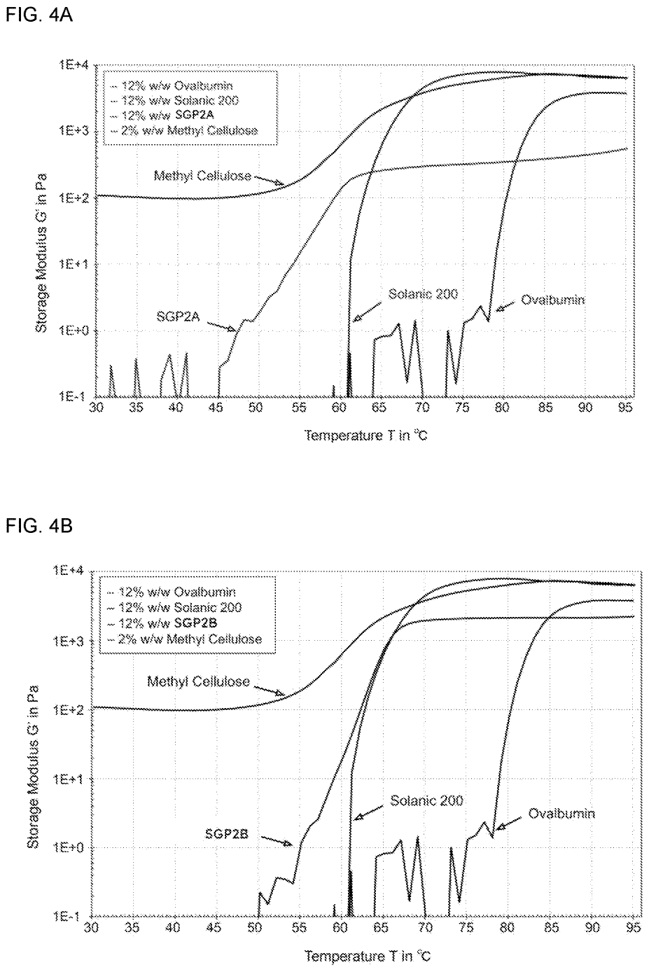

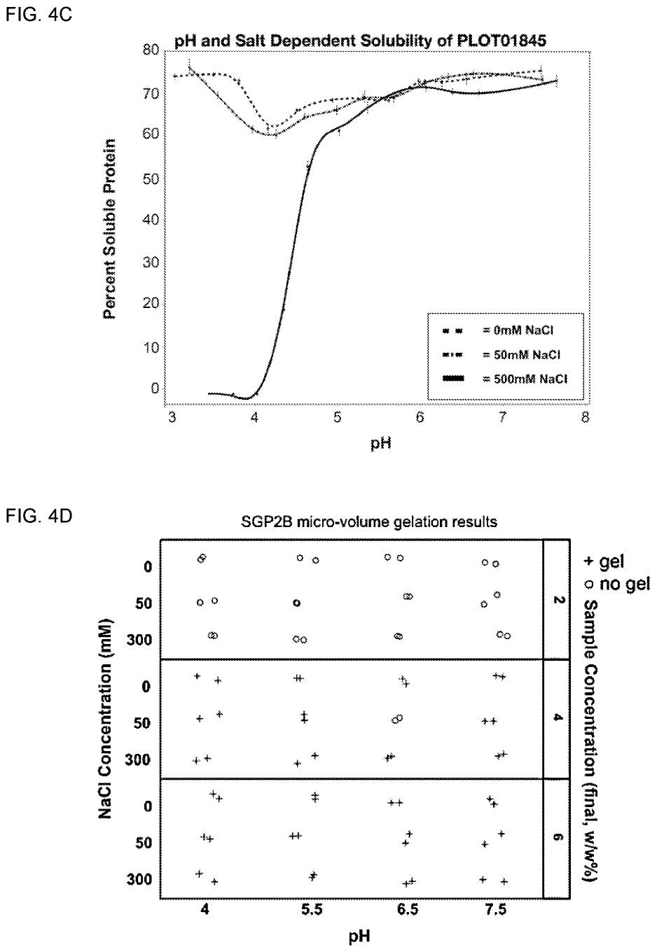

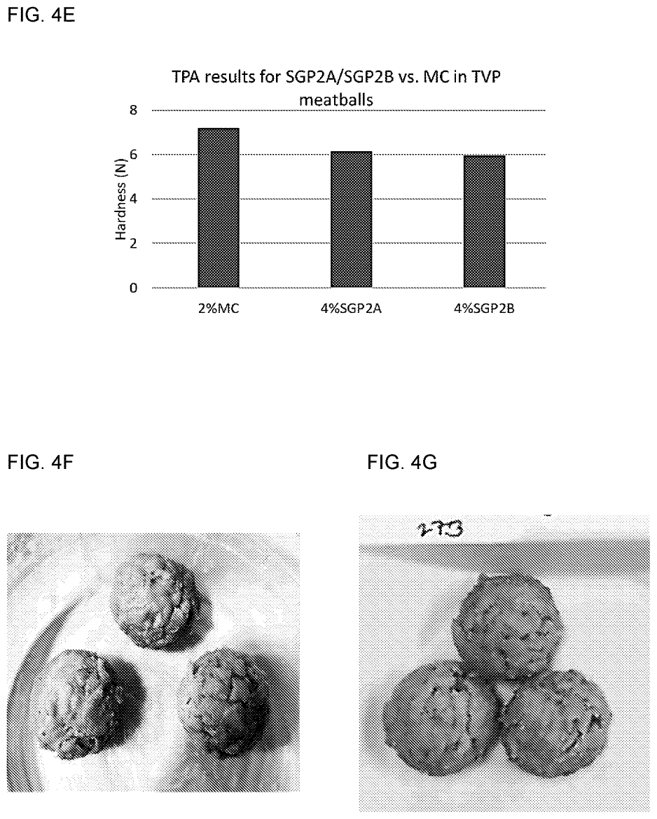

FIG. 4A is a plot of storage modulus (G′) vs temperature for SGP2A, methyl cellulose, Solanic 200 potato protein isolate, and ovalbumin, as measured by small amplitude oscillatory shear rheology. SGP2A shows a large increase in storage modulus (G′), as it undergoes its sol-gel transition during the Phase 1 temperature ramp. The gelation onset temperature, Tₒₙₛₑₜ=50° C., was estimated as the point at which G′ began to rapidly increase above its baseline value at lower temperatures.

Similarly, FIG. 4B is a plot of storage modulus (G′) vs temperature for SGP2B. The protein exhibits a low onset gelation temperature of 55C, estimated as the point where G′ rapidly increased above its baseline at lower temperatures. The gelation onset temperature of 55° C. is slightly higher than that of SGP2A (55° C.) but lower than Solanic 200 potato protein or ovalbumin. SGP2B demonstrates a relatively rapid increase in storage modulus as the solution transitions to a gel in the sol-gel process. As the gel forms and G′ approaches its maximum value during the temperature ramp, the magnitude of the storage modulus for SGP2B's gel is 2241.7 Pa. This approaches a G′ range (103 to 104 Pa) in which the model gelling systems of Solanic 200, ovalbumin, and methyl cellulose also exhibit their maxima.

The relatively low Tₒₛₑₜ of 50° C. is consistent with the low Tm observed in the thermal shift assay. Following the sol-gel transition, a maximum gel strength of 601 Pa was achieved during the temperature ramp, which increased to an ultimate gel strength of 2,127 following Phase 2 (95° C. hold) and Phase 3 (95° C. to 50° C.) of the temperature cycle. The increase in gel strength during cooling is typical of many thermally gelling proteins, and stands in contrast to the “melt-back” phenomenon (a decrease in G′ as the gel is cooled) that is observed with thermoreversible methylcellulose gels.

Following the temperature ramp, a strain sweep measurement was performed in order to probe the integrity of the SGP2A gel superstructure. The strain at which the storage modulus departed the linear viscoelastic region by >5% was defined as the critical strain or “elasticity” of the gel—the point at which the breakdown of the gel structure has started to occur. The ultimate strength of the gel is defined as the value of G′ in the linear viscoelastic region (TABLE 7A and 7B).

Standard commercially used plant-based gelling proteins have critical gelation concentrations in the range of 5% (canola cruciferin) to 18% (pumpkin seed globulins), with critical gelation concentrations most commonly higher than 10% (L. Grosssman et al., Annu. Rev. Food Sci Technol. 2021; 12:93-117). In addition to their generally high critical gelation concentrations, the food-functional performance of plant-based proteins is also commonly restricted by their tendency to exhibit high denaturation temperatures (80° C. to 120° C.) (D. J. McClements et al., Compr. Rev. Food Sci. Food Safety 30 May 2021) and correspondingly high onset gelation temperatures.

FIG. 4C summarizes the effects of pH and sodium chloride concentration on the solubility of SGP2B in 1% wt/vol protein dispersions. The protein concentration detected in the supernatant is expressed as a percentage of the original concentration of protein in the buffered 1% wt/vol starting solution for each data point.

Many plant proteins isolated from seeds and legumes have solubilities below 50% when assessed by similar methods (K. Ma et al., Foods 2022, 11(4):594. The data in FIG. 4C show that the solubility of dispersed SGP2B powder is greater than 60% across all pHs measured in low salt solutions (0 to 50 mM NaCl). Maximum solubilities (>70%) were observed in acidic and alkaline conditions (pH 3 to 3.5, and pH 6 to 8). This indicates that SGP2B protein powder is highly soluble, especially in buffers containing no more than 50 mM salt. At higher salt concentrations (500 mM NaCl), solubility remains high at pH 6 to 8, but sharply declines at pH 4 to 5, which is consistent with aggregate formation.

Because SGP2B is highly soluble at pH 6 to 8, independent of salt concentrations, it is well suited for use in food formulations.

FIG. 4D summarizes gelation propensities of a preparation of SGP2B in an expanded set of food-relevant conditions, surveying a range of protein concentration, salt concentration, and pH. Markers indicate whether the solution formed a gel (+) or remained a sol (o) after thermal cycling. All measurements were performed in duplicate.

Salt- and pH-dependent effects on gelation outcomes are expected to be most pronounced near a protein's critical gelation concentration. At 2% wt/vol of SGP2, gel was not formed under any of the conditions tested. At 4% wt/vol, gel formed in 11 out of 12 conditions tested. At 6% wt/vol, gel formed under all conditions. Thus, the critical gelation concentration is somewhat below 4%. SGP2 demonstrates robust gelation that is insensitive to pH or salt at concentrations of 4% wt/vol or higher.

In contrast to most commonly used plant proteins and plant protein isolates, the combination of low gelation onset temperature (~50° C.) and low critical gelation concentration (4%) means that SGP2A and other GPDHs are suited as a functional animal protein replacement in meat, dairy, and egg alternative products.

### 9. Molecular Mechanism of Gel Formation in Foods and Other Products

The mechanism of the gelling process may be different for different gelation agents, leading to particular attributes. S. Banerjee et al., Crit. Rev. Food Sci. Nutr. 2012; 52:334-346. Some or all of the attributes described in this section can be reproduced using GPDHs, depending on context, and appropriate adjustments in gelation conditions.

Gelatin melts when heated and solidifies when cooled again. Together with water, it forms a semi-solid colloidal gel. Gelation is governed by the partial reformation of triple helices found in collagen during cooling. In the first step, a polypeptide chain takes an orientation to induce a reactive site. Later, condensation of two other chains near the reactive site occur giving rise to triple helix formation. Heat-induced gelation of whey proteins is typical of globular proteins and proceeds through a series of transitions: (i) denaturation (unfolding) of native proteins, (ii) aggregation of unfolded molecules, (iii) strand formation from aggregates, and (iv) association of strands into a network.

Soy proteins are caused to gel by heating soybean flour or milk, followed by addition of salt (Ca⁺⁺ or Mg⁺⁺) to form a gel or curd. Casein molecules are strongly hydrophobic. Sub-micelles are held together by hydrophobic bonds and salt bridges. Enzymatic hydrolysis of k-casein by rennet releases CMP (caseinomacropeptide) and causes the micelles to aggregate leading to rennet gelation. Both ovalbumin and yolk of liquid eggs have the capacity to form gels upon heating. Gel formation is a two-step process of denaturation followed by aggregation of denatured proteins.

Gels are formed from alginates following the addition of polyvalent cations at a low pH (<4). Guluronic acid residues give a buckled conformation providing an effective binding site for the cations. Unlike most other gelling polysaccharides, alginate gels have the particular feature of being cold setting. The gelling characteristics of pectin strongly depend on the degree of esterification. High methoxyl pectins will gel in the presence of sugars or other co-solutes (such as sugars, poly-ols, or monohydric alcohols.

Gelation of agar is a thermo-reversible process and takes place due to the formation of hydrogen bonds. Agar requires heat to bring it into dispersion. On cooling, the hot dispersions set to gels. Molecules undergo a coil helix transition followed by aggregation of helices (Stanley, 2006). Carrageenan is an ionic polymer and forms helical gels on cooling in presence of salts (electrolytes) particularly K⁺ ions. The cations K⁺, Rb⁺, Cs⁺, and NH₄⁺ promote both helix formation and gelation. The gelation of carrageenan generally involves association of the polymer chains by the formation of intermolecular double helices to form ordered domains. Gelation occurs with the subsequent aggregation of these domains mediated by specific binding of the gel promoting cations.

The gelation mechanism of gellan gum is based on the domain model. In aqueous solution at high temperatures, gellan polymers are in a disordered single-coiled state. Cooling of the gellan sol promotes the formation of a threefold left-handed double helix, stabilized by internal hydrogen bonds. Coil-helix conformational transition occurs in a temperature range from 30 to 50° C., depending on the ionic strength of the dispersion. After this transition, the gellan double helixes can be associated in the presence of cations to form junction zones, which can aggregate and lead to the formation of an interconnected three-dimensional gel network, wherein the sol is converted into a gel.

Xanthan gum is caused to gel in the presence of electrolytes over a broad pH range and at high temperatures. Gels are formed on cooling. Xanthan and polymannan chains associate following the xanthan coil-helix transition. Gels from locust bean gum are formed on cooling. Polymannan chains associate following the coil-helix transition, involving galactose deficient regions.

### 10. Screening for Additional Functional and Physicochemical Properties

In surveying and testing other members of the GPDH family, the user will want to assess its gelation characteristics as described above. They may also wish to determine whether a candidate has other desirable functions or properties, thereby increasing the favorability rating of the candidate—and whether it has one or more undesirable functions or properties, thereby decreasing the favorability rating of the candidate or removing it from contention. By way of illustration, desirable properties may include one or more of the following: ease of expression, ease of purification, stability when stored, mixability, and one or more desirable flavors or sensory properties. Undesirable properties may include one or more of the following: allergenicity or immunogenicity, incompatibility with other food ingredients, an adverse physiological effect, and an undesirable flavor.

Computer algorithms are available to predict some of these properties. For example, allergenicity can be predicted in the manner of L. Zhang et al., Bioinformatics 2012, 28:2178-2179; L. Wang et al., Foods 2021, 10:809, doi.org/10.3390; and S. Saha et al., Nucl. Acids Res. 2006, 34, doi:10.1093. Immunogenicity can be predicted in terms of MHG binding motifs and T and B cell epitopes algorithmically in the manner of N. Doneva et al., Symmetry 2021:13, 388. Toxicity can be predicted in the manner of S. S. Negi et al., Sci. Reports 2017:7, 13957-1; and Y. Jin et al., Food Chem. Toxicol. 2017; 109:81-89. Aspects of flavor can be predicted in the manner of P. Keska et al., J. Sensory Studies 2017:e12301; F. Fritz et al., Nucleic Acids Res. 2021 Jul. 2; 49(W1):W679-W684, and S. Ployon et al., Food Chem. 2018 Jul. 1; 253:79-87. Other properties are assessed by laboratory or field testing.

11. Commonly Used Relation Agents in Foods that can be Replaced with GPDHs

GPDHs according to this disclosure can be used to replace gelatin and thickening agents commonly used in food products. Typically used gelation ingredients that may be replaced include methylcellulose, carboxymethylcellulose (CMC), pectin, gums (agar, xanthan, guar, locust bean, k-carrageenan), starches (corn, potato, tapioca), and other proteins (chickpea, pea, fava bean, egg protein, milk protein, wheat gluten, and gelatin).

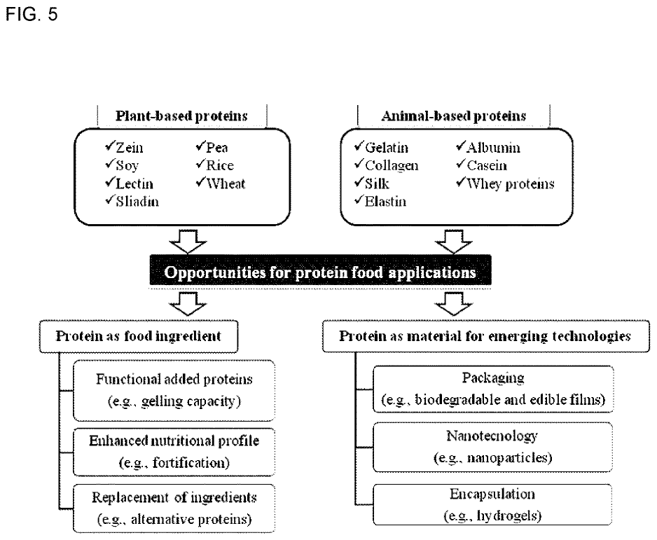

FIG. 5 shows some of the plant and animal proteins currently used in food industry (adapted from JT Martins et al., Front. Sus. Food Sys. 2018, 2:77). Animal-based proteins used in food include gelatins, collagen, silk, elastin, albumin, and milk proteins such as casein, α-lactalbumin, β-lactoglobulin, and lactoferrin. Plant based proteins used in food include zein, soy, lectin, sliadin, pea protein, rice protein, and wheat protein. As food ingredients, these proteins can promote gelation or enhance the nutritional profile of the food product. They can also be used for packaging (in the form of biodegradable or edible films), for formation of nanoparticles, and for encapsulation of food or pharmaceutical agents for delivery.

Hydrocolloids may be incorporated into foods for thickening. D. Saha et al., J Food Sci Technol. 2010 December; 47(6): 587-597. The process of thickening comprises anon-specific entanglement of conformationally disordered polymer chains. Thickening occurs above a critical concentration (the overlap concentration, C*). Below this, the polymer dispersions exhibit Newtonian behavior but show a non-Newtonian behavior above this concentration. Hydrocolloids that have been used as thickening agents in various food systems include starch, modified starch, xanthan gum, galactomannans like guar gum and locust bean gum (LBG), gum Arabic or acacia gum, gum karaya, gum tragacanth, and carboxymethyl cellulose (CMC). The thickening effect depends on the type of hydrocolloid used, its concentration, the food system in which it is used, and also the pH and temperature of the food system.

### 12. Texture Profile Analysis of a Model Food Product

Gelation performance of GDPHs in a food context can be determined by preparing a test food product, and measuring performance characteristics mechanistically.

Test meatballs were prepared using the following ingredients:

TABLE 7C
Ingredients for test meatballs

| | Quantity (% wt/wt) |
|---|---|
| texturized vegetable protein (TVP) | 18.9% |
| water | 37.8% |
| SGP2B | 2.0% |
| water | 18.9% |
| citrus fiber | 0.7% |
| salt | 0.8% |
| coconut oil, melted | 15.0% |
| potato starch | 3.0% |
| soy protein | 3.0% |
| Total | 100.0% |

The procedure for preparing the patties was as follows: Soak TVP in first portion of water for 30 minutes. Dissolve the test protein in second portion of water, add citrus fiber and salt, then emulsify with melted coconut oil. Add potato starch and soy protein to soaked TVP and mix, then add emulsion and mix until fully incorporated. Store mixture at 40° F. for 1 hour. Shape into 10-g meatballs. Bake at 375 F for 8 minutes and verify internal temperature of 165-170 F. Store in an insulated container and perform TPA at 150-155° F.

FIGS. 4E and 4F are images of meatballs created by the aforesaid recipe before and after cooking. Inclusion of SGP2 in the recipe results in a dough that forms the desired shape that is manufacturable at larger scales. The prototype meatballs responded well to cooking, keeping their shape and becoming firm.

The performance characteristics of these prototype patties were objectively measured using the AMETEK™ Brookfield CTX texture analyzer. Texture profile analysis (TPA) was done via a double compression test to 40% deformation at 0.5 mm per sec. using a 5 kg load cell. The readout is the peak force during first compression reported in Newtons (N). Total work (mJ), chewiness (N), gumminess (N), springiness, cohesiveness, and adhesiveness (mJ), can also be determined. Hardness tends to be the most differentiating measurement.

TABLE 7D
Texture profile analysis of meatballs containing 2% SGP2B

| Characteristic | Sample 1 | Sample 2 | Sample 3 | Average |
|---|---|---|---|---|
| hardness (N) | 2.9 | 2.3 | 2.9 | 2.8 |
| sample length (mm) | 24.3 | 24.3 | 23.2 | 23.9 |
| total work (mJ) | 19.3 | 15.8 | 17.3 | 17.4 |
| adhesiveness (mJ) | 0.01 | 0 | 0 | 0 |
| resilience | 0.20 | 0.17 | 0.20 | 0.19 |
| cohesiveness | 0.37 | 0.34 | 0.41 | 0.37 |
| springiness (%) | 0.67 | 0.60 | 0.63 | 0.63 |
| gumminess (N) | 1.80 | 0.79 | 1.19 | 1.02 |
| chewiness index | 0.72 | 0.48 | 0.75 | 0.65 |

FIG. 4G compares the hardness of meatballs containing 4% of recombinant proteins SGP2A or SGP2B with meatballs containing a common binder, 2% methylcellulose. The gelation proteins performed well, causing gelation of the ingredients during cooking, resulting in a cooked product having a desirable final texture.

### 13. Food Products Incorporating GPDHs as Gelation Agents

GPDH proteins are suitable as gelation agents across a range of manufactured food products. The user may incorporate GPDHs into food products at a mass ratio that is appropriate for the degree of gelation they require. This will depend on the other ingredients in the product, whether the product will be heated or otherwise processed by the consumer, and the particular GPDH chosen as the gelation ingredient.

In general, any concentration of between 0.1% and 50% wt/wt of dry food ingredients may be used. A range of 0.5% or 1% to 20% is more typical. When using protein SGP2A or SGP2B, the user may start by testing formulations in a range of 2 or 4 to 12%, to produce the desired network forming, texturizing, and water/oil holding effects in different food systems. Given the improved gel forming propensity of SGP2A in a citrate/phosphate buffer of pH 5.5 to 7.5 supplemented with 50 to 300 mM NaCl, a range of 2 to 10% or 4 to 6% may be appropriate for foods formulated with near-neutral pH and higher salt concentrations. This usage range is comparable to the recommended range for potato protein isolates (2-4%) currently used in popular alternative meat products.

Some specific examples of ranges in foods are the following:
plant-based ground meat: 10 to 20% wt/wt of dry ingredients
plant-based custard or flan: 20 to 30% wt/wt of dry ingredients
vegan cake: 1 to 10% wt/wt of dry ingredients
soup: 20 to 30% wt/wt of dry ingredients
frozen/refrigerated doughs: 10 to 20% wt/wt of dry ingredients

### Plant-Based Patties and Meatballs

SGP2A, SGP2B, and other GPDHs can be used as substitute for gelators and binders in plant-based meats, as illustrated in the following recipes.

TABLE 8A
Ingredients for plant-based beef-like products

| | Quantity (g) | Quantity (% wt/wt) |
|---|---|---|
| Water | 240 | 46.5% |
| Texturized vegetable protein (TVP) | 100 | 19.4% |
| Binder such as methylcellulose, | 6 | 1 to 7% |
| or SGP2A or SGP2B, | | |
| Beet powder (color agent) | 3 | 0.6% |
| Gelling agent such as fava bean | 30 | 0 to 6% |
| protein, or SGP2A or SGP2B | | |
| Garlic powder | 0.9 | 0.2% |
| Nutritional yeast | 6 | 1.2% |
| Dried onion | 0.8 | 0.2% |
| Cocoa powder | 2 | 0.4% |
| Ground thyme | 0.4 | 0.1% |
| White pepper | 1 | 0.2% |
| Malt vinegar powder | 8 | 1.5% |
| Liquid aminos (soy sauce) | 15 | 2.9% |
| Liquid smoke | 1.2 | 0.2% |
| Coconut oil (solidified and beaded) | 100 | 19.4% |

Meat substitutes and flavoring can be made from plant components or other ingredients by combining about 60% (wt/wt) muscle replica (made up, for example, of 62% (wt/wt) dark muscle replica and 38% (wt/wt) white muscle replica), about 30% (wt/wt) fat tissue replica, and about 5% (wt/wt) connective tissue replica. U.S. Pat. No. 10,863,761. Muscle tissue replica can be made by combining a heme binding protein such as myoglobin or leghemoglobin (12 mg/mL) with about an equal volume of plant protein (150 mg/mL) in the presence of a crosslinking agent such as transglutaminase (about 1 wt/vol). Fat tissue replica can be made from moong seed storage 8S globulin or pea globulin by combining with an oil such as soy or rice bran oil in the presence of transglutaminase by heating at ~95° C. for 5 min and then cooling. Fat tissue replica typically forms an opaque gel of off-white color, smooth uniform texture, with no visible discernible liquid that was not incorporated into the gel. Connective tissue replica can be prepared as a combination of plant proteins or structural equivalents that mimic collagen or fascia like fibers, or a combination of the two.

The meat substitute or flavoring may also contain a sugar and/or a sulfur-containing compound that is not part of a protein. The sugar may be selected from glucose, ribose, fructose, lactose, xylose, arabinose, glucose-6-phosphate, maltose, and galactose, and mixtures of two or more thereof. The sulfur-containing compound may be selected from cysteine, cystine, selenocysteine, thiamine, methionine, and mixtures of two or more thereof.

The meat-like flavor or aroma may be manifest during cooking, which results in release of at least two volatile compounds with a meat associated aroma, selected, for example, from 2-methyl-furan, bis(2-methyl-3-furyl)disulfide, 2-pentyl-furan, 3,3′-dithiobis-2-methyl-furan, 2,5-dimethyl-pyrazine, 2-methyl-3-furanthiol, dihydro-3-(2H)-thiophenone, 5-methyl-2-thiophenecarboxaldehyde, 3-methyl-2-thiophenecarboxaldehyde, 2-methyl-thiazole, dimethyl sulfide, decanal, 5-ethyldihydro-2(3H)-furanone, dihydro-5-pentyl-2(3H)-furanone, 2-octanone, 3,5-octadien-2-one, p-Cresol, and hexanoic acid.

Preparation and features of some meat substitutes and flavorings are outlined in U.S. Pat. Nos. 3,815,823; 9,700,067; 10,863,761; 10,798,958; and 11,013,250; and in EP 3952661 A1.

### By Way of Illustration, Meat-Like Patties can be Prepared as Follows;

1. Weigh all dry ingredients
2. Weigh liquid ingredients separately
3. Mix the liquid ingredients with the dry ingredients to form a dough
4. Let the dough hydrate for 20 min at room temperature
5. Make beads of coconut oil: by freezing the coconut oil followed by scraping it with fork, cheese grater or in blender
6. Add half of the coconut oil beads and mix
7. Add the second half of coconut oil beads and gently fold to give a marbled look
8. Plant-based patties cooking procedure:
9. Weigh dough and divide into balls of 50 g
10. Shape them into patties using a patty press maker (2.5″ diameter, measure diameter and weigh the patty again) or shape them into balls for meatballs (50 g each)
11. Cook in non-stick pan (at 150° C. for 5 min per side, flip 3 times or until internal temperature reaches 75° C.)

### Plant-Based Meat-Like Meatballs can be Made as Follows:

1. Preheat oven 400° F.
2. Make balls by hand (25 g each) and place on a baking sheet (use parchment paper)
3. Bake for 20-25 min. Turn meatballs half way into the timer

### Plant-Based Sausages

To make sausages, the following items are combined and packed into a vegetarian casing:

TABLE 8B
Ingredients for plant-based sausages

| | Quantity (% wt/wt) |
|---|---|
| Water | 37.7% |
| Texturized Vegetable Protein (TVP) | 15.7% |
| Sausage binder (Solution of methylcellulose, | 16.6% |
| SGP2A, or SGP2B; 3-8% wt/wt protein | |
| solution in water) | |
| Beet powder | 0.5% |
| Faba bean protein | 4.7% |
| Garlic powder | 0.9% |
| Nutritional yeast | 0.9% |
| Dried onion | 0.1% |
| Cocoa powder | 0.3% |
| Ground thyme | 0.1% |
| white pepper | 0.2% |
| Malt vinegar powder | 1.3% |
| Salt | 1.1% |
| Black pepper | 0.2% |
| Sugar | 0.2% |
| Extra seasoning mix | 0.9 |
| Soy sauce | 2.4% |
| Liquid smoke | 0.2% |
| Coconut oil (solidified and beaded) | 15.7% |

### Preparation is as Follows:

1. Make the sausage binder by mixing methylcellulose, SGP2A or SGP2B with water until completely dissolved
2. Weigh and mix dry and liquid ingredients separately
3. Mix the liquid ingredients with the dry ingredients and the binder to form a dough in the standing mixer at medium speed
4. Let the dough hydrate for 20 min at room temperature
5. Using a sausage filler attachment on a stand mixer, fill the vegetarian casings with the dough and refrigerate or cook on a pan.

### Plant-Based Chicken-Like Nuggets

TABLE 8C
Ingredients for plant-based chicken nuggets

| | Quantity (% wt/wt) |
|---|---|
| Water | 57% |
| Texturized vegetable protein (TVP) | 17% |
| Potato starch | 13-18% |
| Vegetable oil | 3.5% |
| Baking powder | 2.5% |
| Methylcellulose, SGP2A or SGP2B | 1-5% |
| Salt | 0.3% |
| Calcium chloride | 0.2% |

Baking powder and calcium chloride are added to enhance protein water binding capacity and generate the formation of air cells in the dough.

### Preparation is as Follows:

1. Prepare dough by mixing all ingredients in a food processor for 5 min at low speed
2. Mold and shape plant-based nugget dough
3. Steam the molded dough at 100° C. for 14 min
4. Make batter: flour and water in a 1:2 ratio in the quantity required
5. Coat with batter and deep-fry for 1.5 min
6. Freeze at −20° C.
7. Customer preparation: bake at 220° C. for 15 min

### Plant-Based Ice Cream

TABLE 8D
Ingredients for ice cream

| | Quantity (% wt/wt) |
|---|---|
| Water | 60% |
| Soymilk powder or pea protein concentrate | 4.4% |
| Texturized vegetable protein | 8% |
| Safflower oil or vegetable oil | 8% |
| Sweetener (sugar, syrup) | 22.6% |
| Tapioca solids | 2 to 5% |
| Stabilizers SGP2A or SGP2B | 0.2 to 3% |

Protein:fat interaction affects characteristics such as melting rate, stability (mix separation), textural qualities, overrun, and viscosity.

### Preparation is as Follows:

1. Mix soy milk or pea protein powder with water in a saucepan and heat to 45 to 50° C. while continuously stirring
2. Incorporate the rest of the ingredients into the warm mixture while constantly stirring
3. Homogenize ingredients at 15,000 rpm for 3 min
4. Age the mixture at 5° C. for up to 24 h
5. Freeze the mixture while incorporating air (using an ice cream maker) for 20 min

### Plant-Based Flan or Custard

TABLE 8E
Ingredients for flan or custard

| | Quantity (% wt/wt) |
|---|---|
| Unsweetened plant-based milk: coconut, | 80% |
| almond, oat, or soy) | |
| Sugar | 10-16% |
| Vanilla extract | 1% |
| Gelling agents, SGP2A or SGP2B | 3-9% |

Preparation is as Follows. For Flan:
1. Preheat oven to 350° F. (175° C.)
2. For caramel: place 1 cup of sugar into a saucepan over medium-low heat, melt sugar until it liquefies and is a light brown color. While hot, pour syrup into a round baking dish, making sure it is evenly spread
3. Mix all ingredients in a blender until well blended
4. Place the mixture into the baking dish with caramel and cover with aluminum foil. Place the baking dish in a larger secondary container with water (water bath)
5. Bake for 60 minutes
6. Refrigerate until cold
7. Once cold, carefully invert onto a serving plate.

### For Custard:

1. Mix all ingredients in a blender until well blended
2. Cook on the stovetop over medium heat, stirring constantly, until custard thickens
3. Portion into serving dishes and refrigerate until cold

### Vegan Vanilla Cake

TABLE 8F
Ingredients for cake

| | Quantity (% wt/wt) |
|---|---|
| Apple cider vinegar | 1.3% |
| Unsweetened plant-based milk (coconut, | 31-34% |
| almond, oat, or soy) | |
| All-purpose wheat flour | 21-26% |
| Sugar | 22.0% |
| Baking powder | 0.8% |
| Baking soda | 0.3% |
| Salt | 0.6% |
| Vegetable oil | 10.6% |
| Vanilla extract | 2.1% |
| SGP2A or SGP2B solution (1.5-8%) | 8-12% |

### Preparation is as Follows:

1. Preheat the oven to 350° F. (175° C.).
2. Hydrate SGP2A or SGP2B solution in water (concentration may range from 1.5-8%)
3. Mix vinegar and plant-based milk and let stand 5 minutes
4. Mix flour, sugar, baking powder, baking soda, and salt
5. Add oil, vanilla, and protein solution to plant-based milk mixture
6. Add liquid ingredients to dry ingredients and mix until combined
7. Pour batter into an oiled 8-inch cake pan lined with parchment paper
8. Bake for 36-38 minutes or until golden brown and set in the center
9. Let cool at room temperature

### Plant-Based Cookies

TABLE 8G
Ingredients for cookies

| | Quantity (% wt/wt) |
|---|---|
| Flour | 24.9% |
| Salt | 0.3% |
| Baking soda | 0.3% |
| Margarine | 15.5% |
| Sugar | 20-23% |
| SGP2A solution (8-20% in water) | 0.4-2% |
| Vanilla extract | 0.9% |
| Vegan chocolate chips | 37.6% |

### Preparation is as Follows:

1. Preheat oven to 350° F. (175° C.)
2. Hydrate SGP2A in water
3. Cream butter and sugar on medium speed until light in color, about 4 minutes
4. Add SGP2A solution and vanilla extract and mix until well combined
5. Add flour, salt, and baking soda and mix to combine
6. Add chocolate chips and mix briefly
7. Separate into 100 g portions, shape into balls, and place on cookie sheet
8. Bake for 18-21 minutes

### Creamy Mushroom Soup

TABLE 8H
Ingredients for soup

| | Quantity (% wt/wt) |
|---|---|
| Mushrooms (sliced) | 35% |
| Coconut or avocado oil | 3% |
| Vegetable broth | 21-26% |
| Coconut cream | 22-27% |
| SGP2A in water (5 to 15% wt/wt) | 5-10% |
| Onion powder | 1% |
| Garlic powder | 1% |
| Salt | 0.6% |
| Black pepper | 0.6% |

### Preparation is as Follows:

1. In a saucepan over medium-high heat, cook mushrooms in oil for about 5 to 7 min.
2. Add vegetable broth and seasonings, bring to a simmer.
3. Separately, dissolve SGP2A in water (5-15% wt/wt) depending on the desired thickness
4. Stir in coconut cream and protein solution, and cook until the soup has thickened
5. Season to taste with salt and pepper

### Frozen or Refrigerated Dough

SGP2A, SGP2B, and other GPDHs can be used to substitute gums (xanthan, Arabic, methylcellulose, guar) in frozen and refrigerated doughs. P 4,_1

TABLE 8 I
Ingredients for frozen dough

| | Quantity (% wt/wt) |
|---|---|
| Water | 62.4% |
| Wheat flour | 28 to 33% |
| Yeast | 1.5% |
| Salt | 1.6% |
| Margarine (or any other structured fat) | 1.5% |
| SGP2A or SGP2B | 1 to 5% |

Water should be adjusted based on the flour protein concentration and SGP2A or SGP2B. Preparation is as follows.
1. Mix ingredients at low speed until smooth, about 5 minutes
2. Let rest for 10 minutes
3. Divide dough into 150 g portions
4. Knead each portion until structure develops, about 8 minutes
5. Let proof at 28° C. and 85% humidity until the dough volume doubles
6. Freeze quickly
7. Customer preparation: bake at 350° F. for 20-25 minutes

### 14. Regulatory Approval of GPDHs as Food Ingredients

After a particular GPDH has been identified for further development as a food ingredient, the user will assure that all regulatory requirements are met before beginning commercial distribution. For example, new food additives and products thereof for distribution in the U.S. are subject to premarket approval by the Food and Drug Administration (FDA). The new additives are “generally recognized as safe” (GRAS) if there is generally available and accepted scientific data, information, or methods indicating it is safe, optionally corroborated by unpublished scientific data. A notification sent to FDA's Office of Food Additive Safety for approval includes a succinct description of the substance (chemical, toxicological and microbiological characterization), the applicable conditions of use, and the basis for the GRAS determination. The FDA then evaluates whether the submitted notice provides a sufficient basis for a GRAS determination.

15. The Art and Science of Cosmetics, their Preparation and Use

There are thousands of cosmetics on the market, all with differing combinations of ingredients. In the United States alone there are approximately 12,500 unique chemical ingredients approved for use in the manufacture of personal care products. A typical product will contain anywhere from 15-50 ingredients. Considering that some consumers use 9 to 15 personal care products per day, consumers may place as many as 500 individual chemicals on their skin each day through cosmetic use. O. Jones and B. Selinger, Aust. Acad. Sci. 2022.

Most cosmetics contain a combination of at least some of the following core ingredients: water, emulsifier, preservative, thickener, emollient, color, fragrance and pH stabilizers.

Distilled or ultrapurified water forms the basis of almost every type of cosmetic product, including creams, lotions, makeup, deodorants, shampoos and conditioners. It acts as a solvent to dissolve other ingredients and forming emulsions for consistency.

Emulsifiers are used to help keep hydrophilic and hydrophobic components of a preparation from separating. Many cosmetic products are based on emulsions-small droplets of oil dispersed in water or small droplets of water dispersed in oil. Emulsifiers are added to change the surface tension between the water and the oil, producing a homogeneous and well-mixed product with an even texture. Examples of emulsifiers used in cosmetics include polysorbates, laureth-4, and potassium cetyl sulfate.

Preservatives are added to cosmetics to extend their shelf life and prevent the growth of microorganisms such as bacteria and fungi, which can spoil the product and possibly harm the user. Preservatives used in cosmetics are water soluble and non-toxic. They can be natural or synthetic and perform differently depending on the formulation of the product. Some will require low levels of around 0.01%, while other will require levels as high as 5%. Frequently used preservatives include parabens, benzyl alcohol, salicylic acid, formaldehyde and tetrasodium EDTA.

Thickening agents are used to give products an appealing consistency and facilitate use. Lipid thickeners work by imparting their natural thickness to the formula. Examples include cetyl alcohol, stearic acid and carnauba wax. So-called naturally derived thickeners are polymers that absorb water, causing them to swell up and increase the viscosity of a product. Examples include hydroxyethyl cellulose, guar gum, xanthan gum and gelatin. Mineral thickeners absorb water and oils to increase viscosity, but give a different result to the final emulsion than the gums. Popular mineral thickeners include magnesium aluminum silicate, silica and bentonite. Synthetic thickeners are often used in lotion and cream products. The most common synthetic thickener is carbomer, an acrylic acid polymer that is water-swellable and can be used to form clear gels. Other examples include cetyl palmitate, and ammonium acryloyldimethyltaurate.

Emollients soften the skin of the user by preventing water loss. They are used in a wide range of lipsticks, lotions and cosmetics. A number of different natural and synthetic chemicals work as emollients, including beeswax, olive oil, coconut oil and lanolin, as well as petrolatum (petroleum jelly), mineral oil, glycerine, zinc oxide, butyl stearate and diglycol laurate.

Coloring agents and pigments are used in many cosmetics to accentuate or alter a person's natural coloring. Mineral ingredients can include iron oxide, mica flakes, manganese, chromium oxide and coal tar. Natural colors can come from plants, such as beet powder, or from animals, like carmine, often used in red lipsticks. The two most common organic pigments are lakes and toners. The lake pigments are made by combining a dye color with an insoluble substance like alumina hydrate. This causes the dye to become insoluble in water, making it suitable for cosmetics where water-resistant or waterproof properties are desired. A toner pigment is an organic pigment that has not been combined with any other substance. The inorganic metal oxide pigments are usually duller than the organic pigments, but are more resistant to heat and light, providing a longer-lasting color.

Shimmering effects can be created via a range of materials. Some of the most common ones are mica and bismuth oxychloride. The size of the particles used to create pearly and shimmering looks affect the degree of glimmer the product has. The smaller the particle size (15-60 microns, where one micron is one millionth of a meter), the less lustrous the powder will be, and more coverage it gives. Larger particle sizes, up to 500 microns, give a more glittery luster and are more transparent.

Fragrances are often added to liquid and cream cosmetics to improve their appeal.

### 16. Target Properties for Cosmetics, and the Use of GPDHs as Texturizing or Thickening Agents

As a general matter, proteins may be developed for inclusion in cosmetics and other personal care ingredients to impart the cosmetics or ingredients with desired properties, or to enhance the ability of other ingredients to impart such properties. Target properties may include one or more of the following: emulsifying activity, thickness, texture, viscosity, color or color fixing, antibiotic activity, sun protection factor (SPF), water resistance, glossiness, stabilizing activity, moisturizing activity, film-forming, smoothness, lubricity, pearlescence, and physical structuring.

The GPDHs of this disclosure can be used in cosmetics as thickening or texturizing agents. For example, they may be used to enhance the consistency and the touch of finished products. In currently marketed cosmetics, texturizing components are typically polymers (polyacrylates, polysaccharides, or gums) or lipid derivatives (oils, esters, or wax derivatives). Texturizing agents may be used to cause or improve the texture of a product in any manner that is desirable as a process intermediate or final product. For example, the texturizing agent may provide a cosmetic product with creaminess, clarity, thickness, and/or viscosity

The GPDHs of this disclosure can also be used in cosmetics as a thickening agent or thickener that increase the viscosity of a liquid. In some contexts, the GPDH imparts this property without substantially changing its other properties. In other contexts, it imparts the cosmetics with other desirable properties. Thickeners are commonly used cosmetics, and in other industrial products, such as paints, inks, and explosives.

Thickeners may also improve the suspension of other ingredients or emulsions which increases the stability of the product. Thickening agents put forth in this disclosure can be used in cosmetics and personal hygiene products for these and other reasons. Some thickening agents are gelling agents (gellants), forming a gel, dissolving in the liquid phase as a colloid mixture that forms a weakly cohesive internal structure. Other thickeners act as mechanical thixotropic additives with discrete particles adhering or interlocking to resist dispersion or flow when not desired.

The GPDHs of this disclosure can also be used in cosmetics as emulsifiers. Components of cosmetics with emulsifying properties are used in creams and lotions to mix water with oils. There are two types of emulsifiers. Oil-in-water (0/W) emulsifiers keep oil drops packed in water, while water-in-oil (W/O) emulsifiers keep water drops packed in oil. W/O emulsifiers are used for a fatty feel (for example, night & sun protection creams). O/W emulsifiers are used more in moisturizing products (e.g. body lotions, day creams). O/W is the most common type of emulsions in cosmetic preparations. The emulsifying capacity of a water-soluble emulsifier is defined as the maximum amount of oil that can be dispersed in an aqueous solution that contains a specific amount of the emulsifier without the emulsion breaking down or inverting into a water-in-oil emulsion. Emulsifying capacity can also be measured by characterizing the minimum amount of emulsifier required to form an emulsion is to measure the surface load (D), which corresponds to the mass of emulsifier required to cover a unit area of droplet surface

The GPDHs of this disclosure can also be developed to impart cosmetics with other target properties such as those listed above, instead of or as well as their role as texturizers, thickeners, and/or emulsifying agents.

17. Commonly Used Ingredients of Cosmetics and Personal Care Products that can be Replaced with GPDHs

The most commonly used natural gum in currently marketed cosmetics is xanthan gum, which acts as an emulsion stabilizer, film-forming agent and binder. It is obtained by the fermentation of a carbohydrate such as glucose with the bacterium Xanthomonas campestris. Other commonly used ingredients include hydroxyethylcellulose, acacia gum, konjac, sclerotium gum, and hyaluronic acid. Any of these can be substituted with a GPDH as described in this disclosure, Alternatively or in addition, GPDHs can be included in the product as an adjunct to a regularly used ingredient, or to impart additional desirable properties.

Individual proteins can be used in cosmetics to form biopolymers and hydrogels. S. Mitural et al., J. Mat. Sci. (2020) 31:50. Hydrogels are cross-linked networks of macromolecular compounds characterized by high water absorption capacity. Common biopolymer forming agents include collagen, chitosan, hyaluronic acid, and other polysaccharides.

Individual proteins with high water binding and water retaining capacity can be used as alternatives to hyaluronic acid (HA) for topical hydrating formulations. Individual proteins can be used for emulsion stabilizing, viscosifying, or rheology modifying agents in the place of methyl- or ethyl-cellulose and hydroxypropyl methylcellulose. Strong clear protein gels that can be used for moisture retention and physical structuring in topical mask applications. Individual proteins can be used to replace or augment gums/waxes and provide high levels of thickening without tackiness.

Individual proteins can be used as barrier/film forming agents in cosmetics. Ronacare, Merck KGaA, Damstadt, Germany.

Individual proteins can also be used to replace silicones/siloxanes and provide smoothness and lubricity without greasiness. A cyclic methylsiloxane that was once an industry workhorse recently disappeared from the personal care market. Octamethylcyclotetrasiloxane (known as D4) was a relatively inexpensive ingredient that gave skin creams a silky non-greasy feel and hair a luxurious bounce and shine. However, it was subsequently shown that that D4 is potentially toxic and can wash off from hair and skin and build up in the marine environment. This led to replacement of D4 by a related compound: decamethylcyclopentasiloxane, (D5), which has many of the beneficial characteristics of D4. The amount of D4 in cosmetics can range from a few percent by weight to as high as 85% in some hair glosses But now regulators are raising concerns that D5 may also be bio-accumulative, and should be replaced with something else. M. S. Reisch, Chem. Eng. News, 2011.

The GPDHs of this disclosure can be screened and developed to optimize their performance in any of these contexts.

### 18. Regulatory Approval of GPDHs as Ingredients in Cosmetics and Personal Care Products

In the context of this disclosure, the term “personal care product” generally means any article intended to be rubbed, poured, sprinkled or sprayed on, introduced into or otherwise applied to any surface or part of the human body for cleansing, beautifying, promoting attractiveness or altering the appearance, and any item intended for use as a component thereof. Personal care products include cleansing pads, colognes, cotton swabs, cotton pads, deodorant, eye liner, facial tissue, hair clippers, lip gloss, lipstick, lip balm, lotion, makeup, hand soap, facial cleanser, body wash, nail files, pomade, perfumes, razors, shaving cream, moisturizer, baby powder, toilet paper, toothpaste, facial treatments, wet wipes, towels, and shampoo. In the context ofthis disclosure, the GPDH will be a component of a product or ingredient that is a compounded liquid, cream, gel, emulsion, colloid, powder, or dissolvable solid, optionally used in combination with a dispensing agent or personal care device.

Some personal care products and ingredients are regulated by the Food and Drug Administration as cosmetics. The Federal Food, Drug & Cosmetic Act (FD&C Act) defines cosmetics as “articles intended to be rubbed, poured, sprinkled, or sprayed on, introduced into, or otherwise applied to the human body for cleansing, beautifying, promoting attractiveness, or altering the appearance.” Included in this definition are products such as skin moisturizers, perfumes, lipsticks, fingernail polishes, eye and facial makeup preparations, shampoos, permanent waves, hair colors, toothpastes, and deodorants, as well as any material intended for use as a component of a cosmetic product. The U.S. Food and Drug administration characterizes cosmetics as belonging to one of the following categories:
01. Baby Products
02. Bath Preparations
03. Eye Makeup Preparations
04. Fragrance Preparations
05. Hair Preparations (non-coloring)
06. Hair Coloring Preparations
07. Makeup Preparations (not eye)
08. Manicuring Preparations
09. Oral Hygiene Products (dentifrices and mouthwash)
10. Personal Cleanliness
11. Shaving Preparations
12. Skin Care Preparations (Creams, Lotions, Powders, and Sprays); and

### 13. Suntan Preparations

Some personal care products and ingredients are regulated as drugs. The FD&C Act defines drugs as “articles intended for use in the diagnosis, cure, mitigation, treatment, or prevention of disease, and articles (other than food) intended to affect the structure or any function of the body of man or other animals.” Over-the-counter (OTC) drugs are drugs that can be purchased without a doctor's prescription. Certain advertising claims may cause a product to qualify as a drug, even if the product is marketed as if it were a cosmetic. Such claims establish the product as a drug because the intended use is to treat or prevent disease or otherwise affect the structure or functions of the human body. Some examples are claims that products will restore hair growth, reduce cellulite, treat varicose veins, or revitalize cells. Other examples are kin protectants (such as lip balms and diaper ointments), mouthwashes marketed with therapeutic claims, antiperspirants, and treatments for dandruff or acne.

Some personal care products and ingredients meet the FDA definitions of both cosmetics and drugs. This may happen when a product has two intended uses. For example, a shampoo is a cosmetic because its intended use is to cleanse the hair. An antidandruff treatment is a drug because its intended use is to treat dandruff. Consequently, an antidandruff shampoo is both a cosmetic and a drug, because it is intended to cleanse the hair and treat dandruff. Among other cosmetic/drug combinations are toothpastes that contain fluoride, deodorants that are also antiperspirants, and moisturizers and makeup marketed with sun-protection claims. Such products must comply with the requirements for both cosmetics and drugs.

Generally, products categorized as drugs must either receive premarket approval by the FDA or conform to final regulations specifying conditions whereby they are generally recognized as safe and effective, and not misbranded. Cosmetic products and ingredients are not subject to FDA premarket approval authority, with the exception of color additives. Cosmetic firms are responsible for substantiating the safety of their products and ingredients before marketing.

Some personal care products may belong to other regulatory categories, including medical devices (such as certain hair removal and microdermabrasion devices), dietary supplements (such as vitamin or mineral tablets or capsules), or other consumer products (such as manicure sets).

Cosmetic companies may register in the United States through the FDA's Voluntary Cosmetic Registration Program (VCRP). The VCRP assists FDA in carrying out its responsibility to regulate cosmetics. FDA uses the information to evaluate cosmetic products on the market. Because product filings and establishment registrations are not mandatory, voluntary submissions provide FDA with the best information available about cosmetic products and ingredients, their frequency of use, and businesses engaged in their manufacture and distribution (Federal Register 73:76360, and 69:9339).

### 19. Use of GPDHs in Pharmaceutical Manufacture

GPDHs can be used as part of a pharmaceutical or nutraceutical product, for example, by combining with an effective dose of one or more pharmaceutically active agents or nutritional ingredients, optional components such as a pharmaceutically compatible preservative, and an aqueous solvent or excipient. The GPDH will be present at a concentration of 0.5% to 50% or 2 to 20% of GPDH by weight of the final product. The user may wish to adjust the salt and pH of the solution in a way that facilitates formation of the gel at a lower temperature and/or to facilitate dissolution after administration. Since SGP2A and SGP2B come from a plant species, regulatory agencies may be more comfortable using such proteins as part of a pharmaceutical preparation, rather than synthetic gels.

### 20. Regulatory Approval of Pharmaceutical Products

A drug or pharmaceutical product is a composition that contains at least one active agent that requires regulatory approval and provides pharmacological activity or other direct effect in the diagnosis, cure, mitigation, treatment, or prevention of disease, or to affect the structure or any function of the body of man or animals. A nutraceutical product is any substance or ingredient that is promoted as providing a health benefits, but not regulated by the Food and Drug Administration in the U.S.

FDA approval of a drug requires that the drug's effects have been tested for safety and efficacy in clinical trials or their equivalent, and reviewed by the FDA's Center for Drug Evaluation and Research (CDER). The drug is determined to provide benefits that outweigh its known and potential risks for the intended population. The drug approval process takes place within a structured framework that includes:
1. Analysis of the target condition and available treatments-FDA reviewers analyze the condition or illness for which the drug is intended and evaluate the current treatment landscape, which provide the context for weighing the drug's risks and benefits. For example, a drug intended to treat patients with a life-threatening disease for which no other therapy exists may be considered to have benefits that outweigh the risks even if those risks would be considered unacceptable for a condition that is not life threatening.
2. Assessment of benefits and risks from clinical data-FDA reviewers evaluate clinical benefit and risk information submitted by the drug maker, taking into account any uncertainties that may result from imperfect or incomplete data. Generally, the agency expects that the drug maker will submit results from two well-designed clinical trials, to be sure that the findings from the first trial are not the result of chance or bias. In certain cases, especially if the disease is rare and multiple trials may not be feasible, convincing evidence from one clinical trial may be enough. Evidence that the drug will benefit the target population should outweigh any risks and uncertainties.
3. Strategies for managing risks—Risk management strategies include an FDA-approved drug label, which clearly describes the drug's benefits and risks, and how the risks can be detected and managed. In some circumstances, a drug maker may need to implement a Risk Management and Mitigation Strategy (REMS).

### 21. Other Uses of GPDHs in Industrial Products

The ability of SGP2A. SGP2B, and other GPDHs to form robust gels with beneficial properties can be used in other contexts. GPDHs can be used as a storage and/or transport medium, insulator, or packing material for any industrial process that relies or is facilitated by gels.

Proteins can be used as macro-, micro-, or nano-sized delivery vehicles, where an active compound is liberated in a controlled way to the environment when needed. The design of delivery structures depends in part on the protein surface and bulk properties. For example, a delivery device may take advantage of swelling or shrinking capacity when the temperature or pH changes, triggering the release of active compounds. This enhances efficiency, cost-effectiveness, and range of delivery functions, enabling the user to tailor the storage and releasing conditions to the desired outcome.

Thickeners are important components in the paint and printing industries. The products require rheology modifiers to prevent pigments settling to the bottom of the can, yielding inconsistent results. Water based formulas would be nearly impossible with the exception of India ink and the few other water-soluble pigments, but these would have very little coverage and at best would stain wood slightly. All modem paints and inks will have some pigment added at the factory for opacity and to control the specularity of the finish, from matte to high gloss, dependent on thickener used, but more so on the size of the particles added as opacity modifier. Particle sizes of 1 μm and below will be the limit of high gloss, probably confined to luxury automotive coatings, and about 100 μm particulates

In the petrochemical mining industry, gelling agents (solidifiers) are used to react with oil spills, forming rubber-like solids. The gelled coagulated oil then can be removed from the water surface by skimming, suction devices, or nets. In the manufacture of explosives, gelators are used to convert liquid explosives to a gel form. Nitrocellulose and other nitro esters are often used. Many fuels used in incendiary devices also require thickening for increased performance. Gelators in current use include aluminum salts, polystyrene, and hydroxyl aluminum bis(2-ethylhexanoate).

The GPDHs of this disclosure can be screened and developed to optimize their performance in any of these contexts.

In a general sense, the disclosure provides an industrial product for commercial sale or public use that contains a gelation promoting decarboxylase homolog (GPDH) at an effective concentration to cause an adaptation of the product desired by the user. In principle, any industrial product that contains or would benefit from the presence of a gelation, texturizing, thickening, or emulsifying component may benefit by selection and optimization of a GPDH put forth in this disclosure.

### 22. Amino Acid Sequences of SGP2A, SGP2B, and Other GPDHs

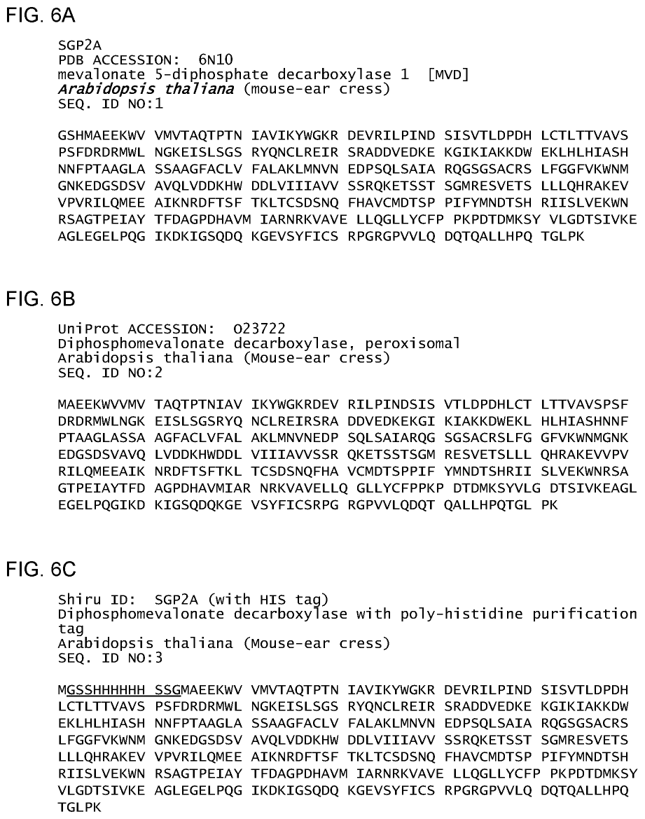

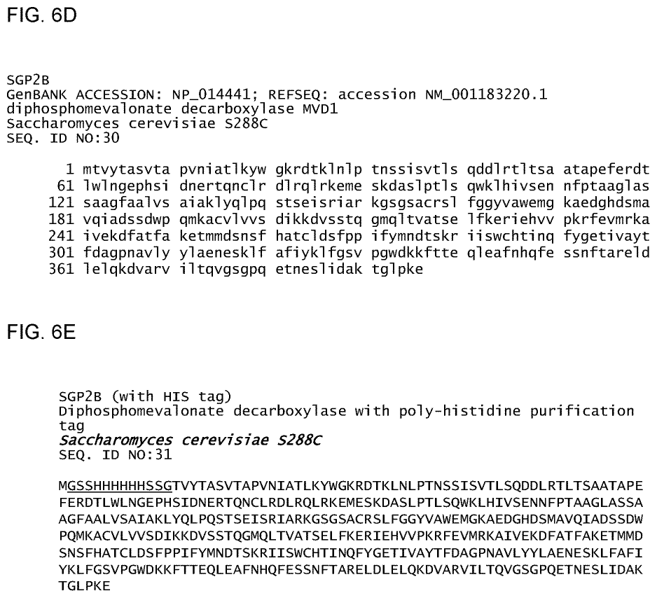

FIGS. 6A to 6C show the amino acid sequence of the prototype GPDH designated as SGP2A (SEQ. ID NOS:1 to 3). FIG. 6A is the sequence initially identified in the protein database PDB by sequence alignment with other proteins believed to have gelling properties FIG. 6B is the sequence of SGP2A obtained from the UniProt database for purposes of expression. FIG. 6C is the protein actually expressed for testing, including minor differences and the addition of a poly-histidine tag at the COOH terminal to facilitate purification.

FIGS. 6D and 6E show the amino acid sequences of a second GPDH prototype designated SGP2B (SEQ. ID NOS: 30 and 31). FIG. 6D is the sequence initially identified by sequence alignment with other proteins believed to have gelling properties. FIG. 6E is the protein actually expressed for testing, including minor differences and the addition of a poly-histidine tag at the COOH terminal to facilitate purification.

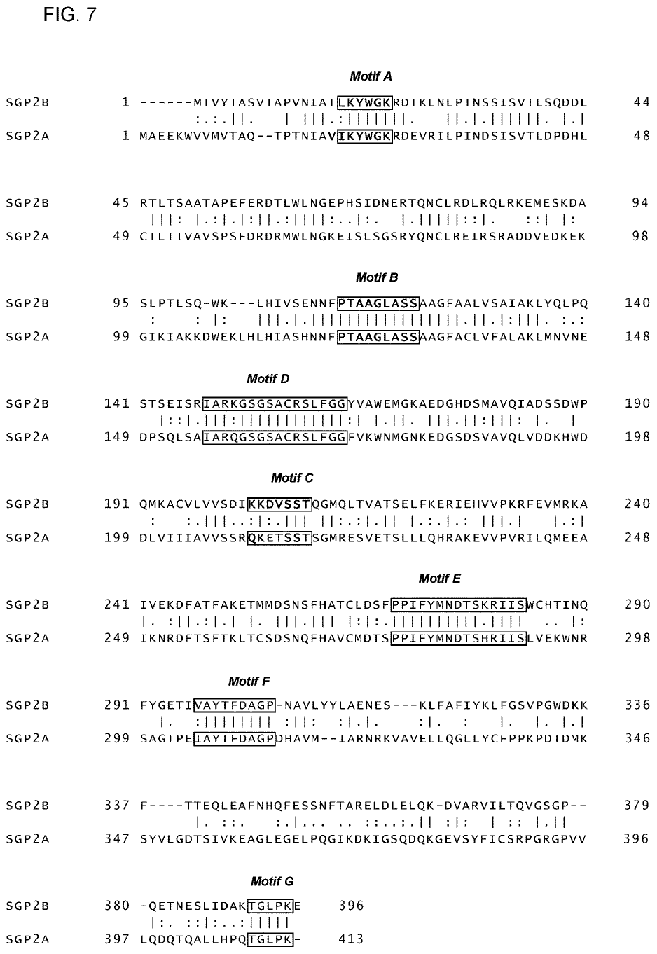

FIG. 7 is a comparison of the amino acid sequences of SGP2A (SEQ. ID NO: 1) and SGP2B (SEQ. ID NO:30). The highlighted regions correspond to the motifs defined below, which are closely conserved between naturally occurring proteins in the GPDH family.

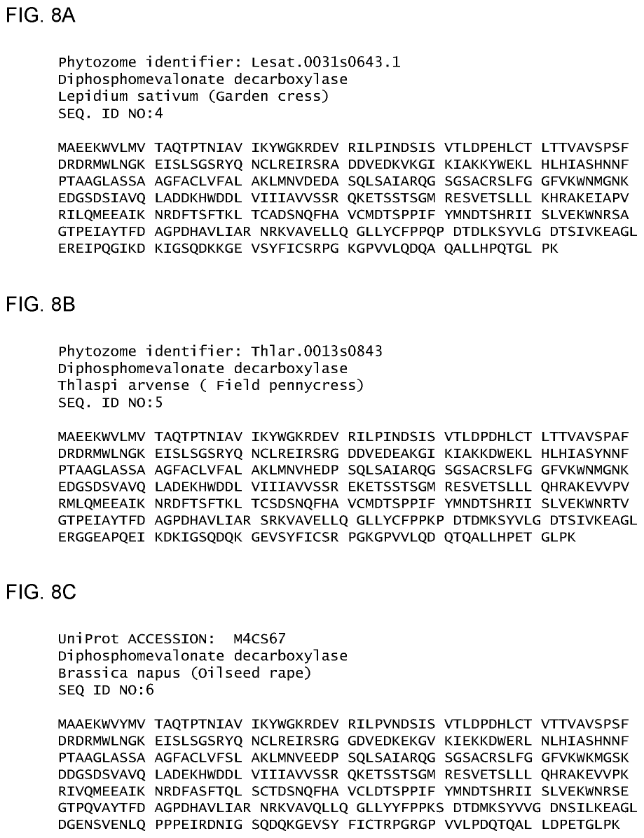

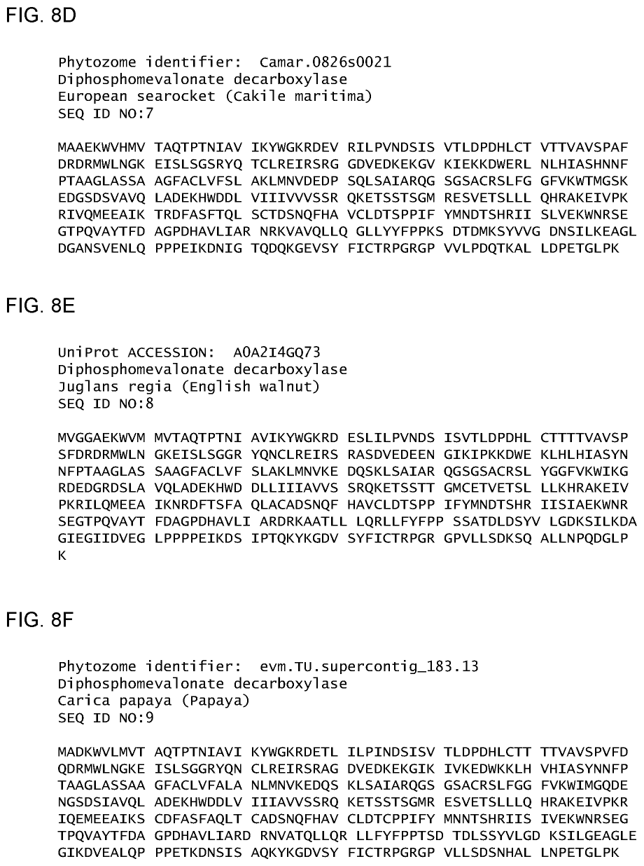

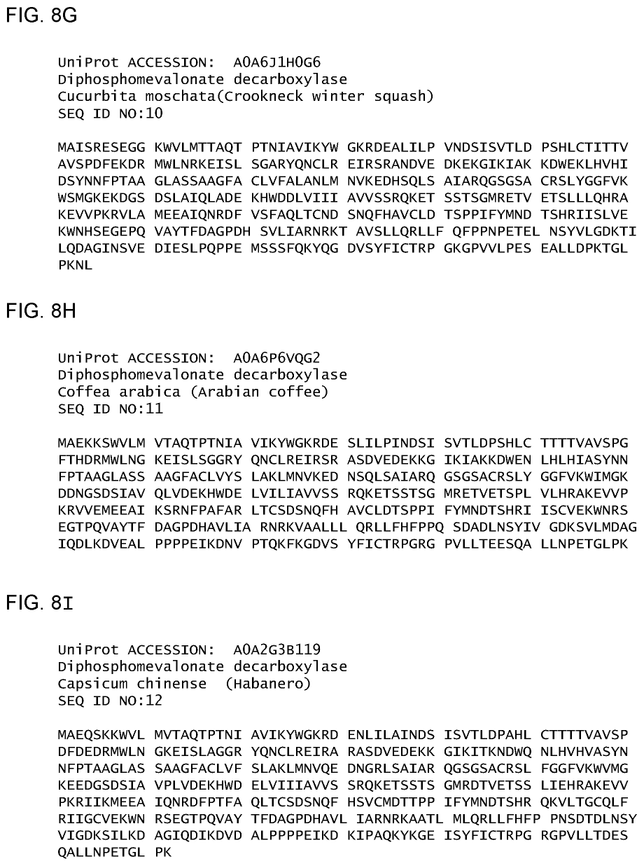

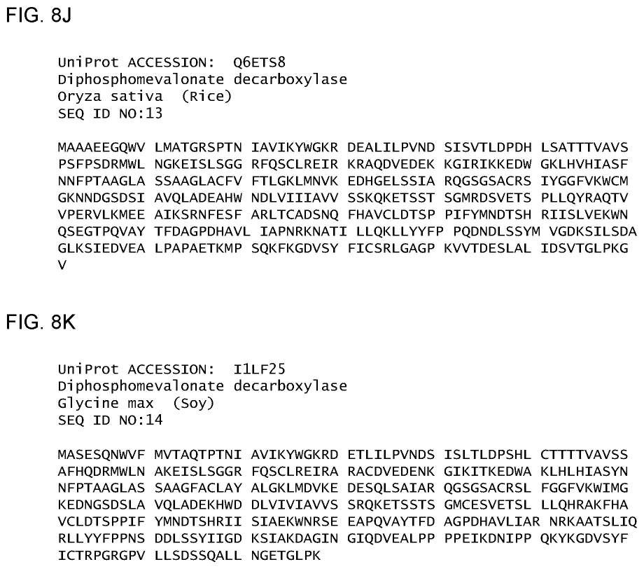

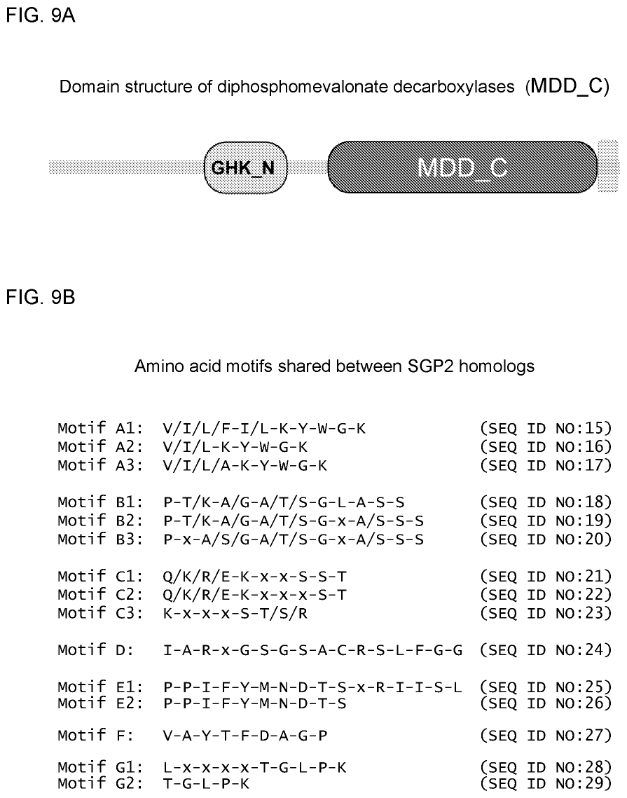

GPDHs that are closely related in sequence identity to SGP2A and SGP2B are also suitable for testing as gelation agents in food products. Strain or species homologs having an amino acid sequence that is at least about 70% identical to the sequence of SGP2A are listed in TABLE 9. The sequences are listed in FIGS. 8A to 8K (SEQ ID NOS:4 to 14).

TABLE 9
Naturally occurring homologs of SGP2A

| SEQ | % identity | | | | UniProt/Phytosome |
|---|---|---|---|---|---|
| ID NO: | to SGP2A | organism | taxon | protein name | identifier |
| 4 | 95.1 | Garden cress | Lepidium sativum | Diphosphomevalonate | Lesat.0031s0643.1 |
| | | | | decarboxylase | |
| 5 | 94.9 | Field | Thlaspi arvense | Diphosphomevalonate | Thlar.0013s0843 |
| | | pennycress | | decarboxylase | |
| 6 | 87.8 | Oilseed rape | Brassica napus | Diphosphomevalonate | M4CS67 |
| | | | | decarboxylase | |
| 7 | 84.5 | European | Cakile maritima | Diphosphomevalonate | Camar.0826s0021 |
| | | searocket | | decarboxylase | |
| 8 | 80.3 | English | Juglans regia | Diphosphomevalonate | A0A2I4GQ73 |
| | | walnut | | decarboxylase | |
| 9 | 79.9 | Papaya | Carica papaya | Diphosphomevalonate | evm.TU.supercontig_{—} |
| | | | | decarboxylase | 183.13 |
| 10 | 78.2 | Crookneck | Cucurbita moschata | Diphosphomevalonate | A0A6J1H0G6 |
| | | winter | | decarboxylase | |
| | | squash | | | |
| 11 | 77.1 | Arabian | Coffea arabica | Diphosphomevalonate | A0A6P6VQG2 |
| | | coffee | | decarboxylase | |
| 12 | 74.8 | Habanero | Capsicum chinense | Diphosphomevalonate | A0A2G3B119 |
| | | | | decarboxylase | |
| 13 | 72 | Rice | Oryza sativa | Diphosphomevalonate | Q6ETS8 |
| | | | | decarboxylase | |
| 14 | 70 | Soy | Glycine max | Diphosphomevalonate | I1LF25 |
| | | | | decarboxylase | |

### 23. Shared Amino Acid Sequence Motifs in GPDHs of Different Species

FIG. 9A shows the domain structure of diphosphomevalonate decarboxylases that is shared with the homologs of SGP2A and SGP2B listed in TABLE 9. Beginning at the N-terminal, there is a GHMP kinases N terminal domain (“GHK N”), followed by a mevalonate 5-diphosphate decarboxylase C-terminal domain (“MDD_C”) and a short disordered region. The GHK N domain is structurally conserved in eukaryotes and prokaryotes, and proteins that include this domain are kinases involved in multiple key metabolic pathways.

FIG. 9B shows amino acid motifs (SEQ ID NOS:15 to 29) that were identified during the course of this project as shared features of the sequences shown in these alignment.

Motifs A1, A2, and A3 come from and help identify the GHMP kinase N′ domain. Motifs B1, B2, B3, as well as motifs C1, C2 and C3 come from and help identify the mevalonate 5-diphosphate decarboxylase C-terminal domain. The amino acids highlighted in light gray represent residues that are involved in the catalytic cycle of members of this enzyme family, and the lysine shaded in black represents the catalytic residue that is critical to the native function of diphosphate decarboxylase enzymes.

Motifs A1, B1, and C1 were determined using a sequence alignment that was generated in the course of a project to characterize certain features of candidate GPDHs. The alignment is shown in the U.S. provisional application to which this disclosure claims priority. The amino acid sequences of thirty five proteins were extracted from the Pfam protein database build 35.0 (November 2021, 19632 entries), available from the European Molecular Biology Laboratory. J. Mistry et al., Nucleic Acids Research (2020) doi: 10.1093/nar/gkaa913. The extracted sequences were identified in Pfam as having sequence patterns (hidden Markov models) corresponding to both a GHMP kinase N′-terminal domain (PF00288) and the mevalonate 5-diphosphate decarboxylase C-terminal domain (PF18376). The sequences were aligned using the fast Fourier transform algorithm MAFFT. K Katoh et al., Nucl Acids Res. 2002; 30:3059-3066. All sequences were identified as having a significant similarity to the SGP2A or SGP2B sequence via the BLAST algorithm (SF Altschul et al., 1990; J. Mol Biol. 2015:403-410).

Motifs A2, B2, and C2 were determined using a sequence alignment of the amino acid sequences of another forty two candidate GPDHs. These proteins were extracted from the UniProtKB database (UniProt Consortium, 2021; Nucl. Acids Res. 215:403-410) as having significant hits of the GHMP kinase N′-terminal domain (PF00288) and the mevalonate 5-diphosphate decarboxylase C-terminal domain (PF18376) PFAM domains. All sequences were identified as having a significant similarity to the SGP2A or SGP2B sequence via the BLAST algorithm (SF Altschul et al., 1990; J. Mol Biol. 2015:403-410).

Motifs A3, B3, and C3 were determined using a sequence alignment of the amino acid sequences of another sixty candidate GPDHs. These proteins were extracted from the UniProtKB database (UniProt Consortium, 2021; Nucl. Acids Res. 215:403-410) as having significant hits of the GHMP kinase N′-terminal domain (PF00288) and the mevalonate 5-diphosphate decarboxylase C-terminal domain (PF18376) PFAM domains. All sequences were identified as having a significant similarity to the SGP2A or SGP2B sequence via the BLAST algorithm (SF Altschul et al., 1990; J. Mol Biol. 2015:403-410).

Motifs D, E1, E2, F, G1, and G2 were identified by comparing the amino acid sequences of SGP2A and SGP2B shown in FIG. 7.

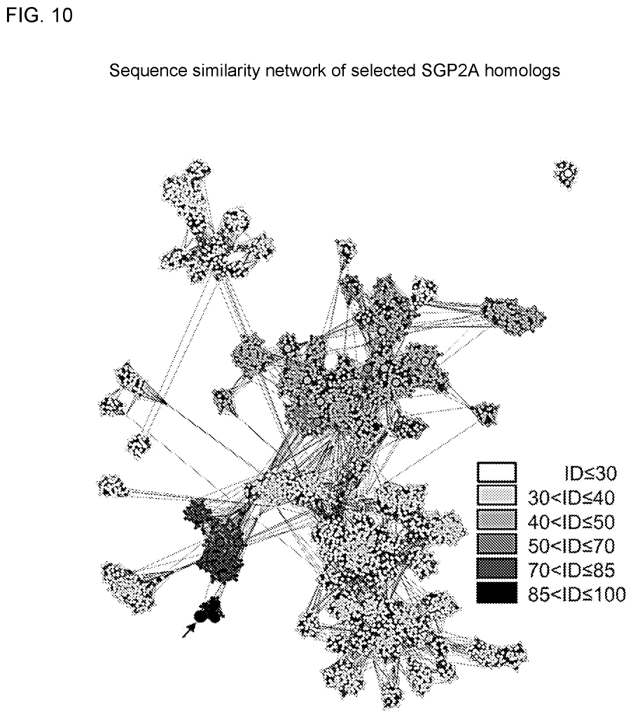

FIG. 10 is a sequence similarity network of selected GPDHs. This a graphical representation of the degree of pairwise sequence identity between different diphosphomevalonate decarboxylases, where each circle represents a particular sequence. The length of the line between sequences reflects the degree of sequence similarity. Sequences for the network were collected by gathering all UniProtKB sequences that match both the GHMP kinases N terminal and the mevalonate 5-diphosphate decarboxylase C-terminal sequence patterns, as defined by PFAM. Larger nodes represent proteins characterized as diphosphomevalonate decarboxylases, as annotated in SwissProt. Edges represent pairwise sequence similarity as measured by BLAST with bitscore higher or equal to 100 (equivalent to ~30% sequence identity). The nodes are density coded by the percent identity between the protein sequence represented by the node and protein X (as depicted in the legend).

### 24. Exclusive Nature of GPDHs Having One or a Combination of the Aforesaid Motifs

Proteins having similar functions often have homologs and isologs that are closely related in amino acid sequence across a range of species. The GPDH family is unusual, in the sense that species and strain homologs may have a sequence identity as low ss 40% (FIG. 7). Even so, the naturally occurring proteins generally have substantially the same domain structure and share amino acid motifs that are closely conserved across the plant kingdom. For this reason, the definitions for GPDHs given and claimed in this disclosure are quite conservative, and encompass a very modest number of related proteins.

This can be illustrated by using the structural definitions put forth in the previous section to query a large protein database.

Shiru (the owner of this invention) has assembled its own database of over 400 million protein sequences at the time of this writing, culled from a number of public and private databases. A subset of the database (the Plant+ subset) are proteins that originate in plants, fungi, and cyanobacteria.

TABLE 10
Proteins in database having structural features of GPNs

| | Number of proteins | |
|---|---|---|
| | in the entire | Number in Plant+ |
| | database with this | subset with this |
| Criteria | criteria | criteria |
| 40% identical to P103 | 5,108 | 1,926 |
| Motif A1 | 9,724 | 1,141 |
| Motif A2 | 16,604 | 1,834 |
| Motif A3 | 17,189 | 1,916 |
| Motif B1 | 9,067 | 1,706 |
| Motif B2 | 18,050 | 2,867 |
| Motif B3 | 140,641 | 15,862 |
| Motif C1 | 556,323 | 85,256 |
| Motif C2 | 6,059,696 | 709,345 |
| Motif C3 | 71,807,791 | 8,284,687 |
| Motif D | 1,214 | 948 |
| Motif E1 | 413 | 305 |
| Motif E2 | 1,046 | 763 |
| Motif F | 2,246 | 651 |
| Motif G1 | 21,710 | 1,548 |
| Motif G2 | 3,365 | 591 |
| Motifs A2 + B1 + C1 | 2,094 | 1,184 |
| Motifs A2 + B1 + C1 + E1 + | 186 | 150 |
| G2 | | |
| Total number of sequences | 446,680,429 | 27,355,612 |
| in database in the database | | |

These data show that GPDHs are very rare and selective proteins: less than 0.01% of all proteins in the Plant+database have any one of the Motifs A1, A2, A3, B1, B2, D, E1, E2, F, and G2. Even fewer have one of these Motifs in combination with others. Because of the shared motifs, SGP2A and SGP2B are representative of the genus.

The numbers in TABLE 10 are modest compared with definitions of amino acid homologs often presented in patent disclosures. For example, if a protein is identified as having 90% sequence identity to a sequence of 100 consecutive amino acids, there are (100!/10!/90!) combinations of positions that may vary, each by 20 amino acids. The total number of possible sequence variants within the 10% limitation would be roughly 3.5×10¹⁴.

### 25. Optional Changes to Amino Acid Sequence to Further Optimize Performance of GDPH Proteins

In some instances, the naturally occurring protein upon which a GDPH is based may have an enzyme activity or binding affinity that may be deemed unfavorable for human consumption. In this case, the user has the option of adapting the naturally occurring protein or portion thereof by altering its amino acid sequence to remove such activity or to add or delete a glycosylation site. The altered form can be designed empirically, for example, by random mutation of the native sequence or portion, and testing the functional properties of the altered protein. Alternatively, the altered form may be rationally designed with reference to the known three-dimensional structure of the protein and its suspected functional domains, making one, two, three, five, ten, or more than 10, or between 1 and 5,1 1 and 10, or 1 and 25 amino acid changes in the form of substitutions, additions, or deletions that remove residues essential to the unwanted binding or catalytic site, or cause refolding of the protein so that the enzyme substrate or an enzyme cofactor does not bind.

### 26. Definition and Scope of the GPDH Family

The term “gelation promoting decarboxylase homolog” (“GPDH”) as used in this disclosure refers to a family of proteins bearing structural resemblance to the prototype gelation SGP2A (SEQ. ID NO: 1) or SGP2B (SEQ ID NO:30). For the purposes of this disclosure, a protein falls within the definition of a GPDH if it has one or more of the structural characteristics referred to below, and has a measurable ability to create a gel or cause gelation or thickening of a mixture of food ingredients in which it is included. Beyond promoting gel formation, individual GPDHs of this disclosure may or may not have enzyme activity.

The initialism GDPH is used in this disclosure convenience only, and implies nothing about the function, characteristics, or physiological role of a GPDH family member, unless explicitly stated otherwise. Some GPDHs are naturally occurring, which means that they are produced by living organisms that have not been genetically modified with respect to the GPDH encoding gene. GPDHs may be full-length gene products, including splice variants, or they may be fragments of a gene product produced in the normal course of expression and operation. They may have either or both of the domains shown in FIG. 9A. Optionally, the user may create artificial fragments, fusion proteins, and amino acid variants that fall within the same structural and functional definition of a GPDH.

Unless stated otherwise, GPDHs for use according to this disclosure have one or more of the following structural features:
They have an amino acid sequence that contains at least one motif selected from A1, A2, and A3, and/or at least one motif selected from B1, B2, and B3, and/or at least one motif selected from C1, C2, and C3, and/or at least one of motifs D, E1, E2, F, G1, and G1 as shown in FIG. 9B;
They have an amino acid sequence that is at least 60%, usually at least 70% (and possibly at least 80%, 90%, 95%, OR 98.5% identical to any one or more of SEQ. ID NOS:1 to 14 and 30 to 31, as determined by the BLAST algorithm (SF Altschul et al., 1990; J. Mol Biol. 2015:403-410).

Besides sharing motifs and/or other structural features with SGP2A and/or SGP2B, a GPDH of this disclosure also has the property of promoting gelation or thickening of a food comprising a mixture of food ingredients that include the GPDH, either at the time of manufacture or when the food is cooked or otherwise processed by the consumer.

A GPDH is characterized as promoting gelation of a product composing multiple ingredients if it increases the gelling characteristics of the product during formulation in relation to a product with the same ingredients except for the GPDH. Alternatively or in addition, it may promote gelation of the product after heating beyond the Tₒₙₛₑₜ and then cooling, compared with the same product before heating. This characterization may be made at any concentration between 0.1% and 20%, (or between 1% and 12%, or at 5% or 12%) of all ingredients in the product by dry weight. Quantitatively, the storage modulus of the product will be increased by at least 200, 500, 1,000, 5,000, or 10,000 Pascals (Pa).

A GPDH is characterized as causing thickening of a product composing multiple ingredients if it increases the gelling characteristics of the product during formulation in relation to a product with the same ingredients except for the GPDH. Alternatively or in addition, it may cause thickening of product after heating beyond the Tₒₙₛₑₜ and then cooling, compared with the same product before heating. This characterization may be made at any concentration between 0.1% and 20%, (or between 1% and 10%, or at 5%) of all ingredients in the product by dry weight. Quantitatively, the viscosity of the product will be increased by at least 100, 200, 500, 1,000, or 2,000 centipoise (cP).

### 27. Incorporation by Reference

Each and every publication and patent documents cited in this disclosure are hereby incorporated herein by reference in their entireties for all purposes to the same extent as if each such publication or document was specifically and individually indicated to be incorporated herein by reference.

### 28. Interpretation and Implementation

Although the technology described above is illustrated in part by certain concepts, procedures, and information, the claimed invention is not limited thereby except with respect to the features that are explicitly referred to or otherwise required. Theories that are put forth in this disclosure with respect to the underlying mode of production, action, and assessment of various products and components are provided for the interest and possible edification of the reader, and are not intended to limit practice of the claimed invention.

While the GPDHs described in this disclosure were developed by the inventors and the owner of this technology as gelation causing agents with superior properties, SGP2A, SGP2B, and other GPDHs referred to in the claims that follow may be used in the manufacture of food for any reason, including but not limited to gelation. Information about the physiological role of SGP2A, SGP2B, and other GPDHs is historical, and does not limit the use of SGP2A, SGP2B, their homologs or any other product that falls within the definition of a GPDH as a food ingredient unless explicitly stated otherwise. The reader may use the technology put forth in this disclosure for any suitable purpose.

Although aspects of the invention has been described with reference to the specific examples and illustrations, changes can be made and substituted to adapt to a particular context or intended use as a matter of routine development and optimization and within the purview of one of ordinary skill in the art, thereby achieving benefits of the invention without departing from the scope of what is claimed below and equivalents thereof.

1-33. (canceled)

34. A method of causing gelation or thickening of a commercial product or component thereof during manufacture or upon use, the method comprising including in or adding to the product or component a gelation promoting decarboxylase homolog (GPDH) at a concentration of 1 to 20% by weight of dry ingredients,
wherein the GPDH is a protein or protein fragment that is naturally expressed by a non-animal organism;
wherein the GPDH has an amino acid sequence that contains Motif A3 (SEQ ID NO: 17), Motif B3 (SEQ. ID NO:20), and Motif C3 (SEQ. ID NO:23); and/or
wherein the GPDH has an amino acid sequence that is at least 80% identical to SGP2A (SEQ. ID NO: 1) or SGP2B (SEQ. ID NO:30).

35. The method of claim 33, which is a method of causing gelation or thickening of a food product during manufacture or upon heating, the method comprising including in the food product said purified or recombinant GPDH at a concentration of 1 to 20% by weight of dry ingredients in the food product.

36. The method of claim 35, wherein the food product is prepared using a recipe in which one or more gelation or thickening agents previously used in such product is replaced with said GPDH,
wherein the gelation or thickening agent that is replaced by the GPDH is one or more of the following: methylcellulose, carboxymethylcellulose (CMC), pectin, xanthan gum, guar gum, locust bean gum, carrageenan, starch, tapioca, pea flower, fava bean flower, egg, milk, wheat protein, and gelatin.

37. The method of claim 35, wherein the food product is a meat substitute in the form of patties, meatballs, sausage, or chicken-like nuggets, plant-based ice cream or other dairy substitute, a flan or custard, a cake, a soup, or frozen or refrigerated dough.

38. The method of claim 35, wherein the food product is a meat substitute or flavoring that also comprises:
a) a protein content of at least 10% by weight, wherein at least 75% of the protein content is a mixture of plant proteins and/or one or more product of tissue culture; and
b) a fat content of at least 5% by weight, wherein at least 75% of the fat content is one or more plant derived oils; and optionally
c) 0.2% to 5% by weight of a heme-containing protein or a porphyrin binding protein;
wherein after cooking, the food product or a product made from the flavor additive has a meat-associated aroma and/or taste; and/or
wherein the protein content and the fat content form a muscle replica and a fat tissue replica that are assembled in the product in a manner that approximates the physical organization of meat.

39. The method of claim 35, wherein the food product is a plant based ice cream that also comprises:
a) a protein content of at least 5% by weight, wherein at least 75% of the protein content is a mixture of plant proteins and/or one or more products of tissue culture; and
b) a fat content of at least 5% by weight, wherein at least 75% of the fat content is one or more plant derived oils; and
c) a sweetener of at least 5% by weight;
which when frozen at −5 to −25° C. stays mixed and has the mouthfeel of an ice cream.

40. The method of claim 33, which is a method of texturizing, thickening, or emulsifying a cosmetic product or personal care ingredient during manufacture, the method comprising including in the product or ingredient said purified or recombinant GPDH at a concentration of 1 to 10% by weight of the product or ingredient.

41. The method of claim 40, wherein the cosmetic product or personal care ingredient prepared using a formulation in which one or more components previously used in such product is replaced with said GPDH,
wherein the component that is replaced by the GPDH is one or more of the following: hyaluronic acid (HA), methyl- or ethyl-cellulose, hydroxypropyl methylcellulose, a gum, or a wax.

42. The method of claim 40, wherein the cosmetic product or personal care ingredient is a moisturizer, eye or skin makeup preparation, lipstick, lip balm, lotion, facial cleanser, pomade, shaving cream, oral hygiene product, facial treatment, skin care preparation, or a suntan or sunblock preparation.

43. The method of claim 33, wherein the GPDH contains Motif A1 (SEQ ID NO: 15), Motif B1 (SEQ. ID NO: 18), and Motif C1 (SEQ. ID NO:21),

44. The method of claim 33, wherein the GPDH further contains any one or more of Motif D (SEQ ID NO:24), Motif E2 (SEQ. ID NO:26), Motif F (SEQ. ID NO:27), and Motif G (SEQ. ID NO:29).

45. The method of claim 33, wherein the GPDH contains Motif A3 (SEQ ID NO: 17), Motif C3 (SEQ. ID NO:20), Motif E (SEQ. ID NO:26), and Motif G2 (SEQ. ID NO:29).

46. The method of claim 33, wherein the GPDH is at least 80% identical to SGP2A (SEQ. ID NO:1)

47. The method of claim 33, wherein the GPDH is at least 80% identical to SGP2B (SEQ. ID NO:30).

48. The method of claim 33, wherein the naturally occurring form of the GPDH has diphosphomevalonate decarboxylase enzyme activity.

49. The method of claim 33, wherein the amino acid sequence of the GPDH has been adapted from its naturally occurring sequence with one to ten amino acid substitutions, additions and/or deletions.

50. The method of claim 33, wherein said GPDH has one or more of the following properties:
a critical gelation concentration of no more than 6%.
a Tₒₙₛₑₜ of 40 to 70° C., and/or
an ultimate gel strength of at least 2,000 Pa.

51. The method of claim 33, wherein said GPDH is produced by recombinant expression.

52. A food product, food additive, cosmetic, or an ingredient of a personal care product that contains a gelation promoting decarboxylase homolog (GPDH) at a concentration of 1 to 10% by weight of dry ingredients,
wherein the GPDH is a protein or protein fragment that is naturally expressed by a non-animal organism and has an amino acid sequence that is at least 80% identical to SGP2A (SEQ. ID NO:1),
wherein said food product, additive, cosmetic, or ingredient contains less than 2% by weight of other proteins naturally expressed by said organism.

53. A food product according to claim 52, which comprises:
a) said GPDH, produced by recombinant expression;
b) a content of other proteins at least 10% by weight, which is a mixture of plant proteins;
c) a fat content of at least 5% by weight, comprising one or more plant derived oils; and optionally
d) a sweetener.
